(19) United States
(12) Patent Application Publication
(10) Pub. No.: US 2011/0077731 A1
(43) Pub. Date: Mar. 31, 2011

(54) MULTIPLE STENT DELIVERY SYSTEM

(75) Inventors: Henry H. Lee, San Diego, CA (US); Kevin M. Seiki, Murrieta, CA (US); John Whitfield, Temecula, CA (US); John Papp, Temecula, CA (US); Benjamin Huter, Murrieta, CA (US)

(73) Assignee: ABBOTT CARDIOVASCULAR SYSTEMS INC., Santa Clara, CA (US)

(21) Appl. No.: 12/567,429

(22) Filed: Sep. 25, 2009

(51) Int. Cl. A61F 2/06; A61M 31/00

(52) U.S. Cl. 623/1.11; 604/500

(57) ABSTRACT

A stent delivery system having multiple stents in a single delivery catheter, configured for delivering and deploying at least some of the stents in a patient's anatomy. A reversibly collapsible stent stop secured to the inner tubular member abuts a proximal end of a first distal stent prior to deployment of the first stent, and is configured to radially collapse as the inner tubular member is proximally withdrawn into the outer tubular member through the second collapsed stent, and radially self expand along at least a section thereof at a location proximally adjacent to a proximal end of a second collapsed stent prior to deployment of the second stent after deployment of the first stent. One or two stent retainer are attached to the shaft of a stent delivery catheter to prevent longitudinal shifting of the stent along the longitudinal axis of the catheter.

### BACKGROUND OF THE INVENTION

This invention relates generally to catheters, and more particularly to catheter systems for percutaneous transluminal procedures, such as delivery and deployment of expandable prostheses.

In the treatment of vascular and biliary disease, expandable endoprosthesis devices, generally called stents, are commonly implanted into a patient's body lumen to maintain the patency thereof. Stents are particularly useful in the treatment and repair of body lumens after a stenosis has been compressed by percutaneous transluminal coronary angioplasty (PTCA) or percutaneous transluminal angioplasty (PTA), or removed by atherectomy or other means, to help improve the results of the procedure and reduce the possibility of restenosis. Stents are generally cylindrically shaped devices which function to hold open a segment of a blood vessel such as a coronary artery, peripheral artery, or other body lumen such as a bile duct. Stents are usually delivered in a collapsed state on a catheter to the target site and then deployed at that location by expanding to a larger diameter into contact with the body lumen wall. Stents are generally classified into one of two categories related to the expansion of the stent, namely, stents which require application of a radially outward force such as by inflating a catheter balloon on which the stent is mounted, or alternatively, self-expanding stents which will automatically expand from the collapsed state when the stent is advanced out the distal end of a radial restraining member of the delivery catheter.

Prior art stent delivery systems for implanting self-expanding stents typically include an inner lumen around which the collapsed stent is positioned and an outer restraining sheath which is initially placed over the collapsed stent prior to deployment. When the stent is to be deployed in the body vessel, the outer sheath is moved in relation to the inner lumen to uncover the collapsed stent, allowing the stent to expand to its expanded condition. Delivery systems have utilized a push-pull type technique in which the outer sheath is retracted while the inner lumen is pushed forward, or have been designed to retract the outer sheath and deploy the stent while the inner lumen must remain stationary to prevent the stent from moving axially within the body lumen during deployment.

It is important in any typical vascular intervention procedure to have a relatively short intervention duration, and to have uncomplicated steps which are relatively few in number. This results in a lowered potential for complications. For example, an extended intervention time increases the risk of infection or stress on the patient, and a large number of device insertions and removals increase the potential for creation of emboli which can cause stroke or small vessel occlusions distal to the intervention site. However, one difficulty has been providing a device which facilitates accurately delivering multiple stents within a patient quickly and easily, such that the procedural steps required to use the device are relatively simple and straight forward.

### SUMMARY OF THE INVENTION

The invention is directed to a stent delivery system having multiple stents in a single delivery catheter, configured for delivering and deploying at least some of the stents in a patient's anatomy.

In one aspect of the invention, the stent delivery system generally includes a delivery catheter having an inner tubular member and an outer tubular member adapted for axial movement with respect to each other, a first stent and at least one second proximal stent in a collapsed configuration in a space between the inner tubular member and outer tubular member within the delivery catheter and configured to radially self expand from the collapsed configuration to an expanded configuration upon removal of a radially restraining force of the catheter outer tubular member, and a reversibly collapsible stent stop secured to the inner tubular member. The outer tubular member has an advanced configuration surrounding a distal section of the inner tubular member, and a proximally retracted configuration, and the inner tubular member is configured for proximally withdrawing the distal section of the inner tubular member into the outer tubular member in the retracted configuration and thereby transitioning the outer tubular member from the retracted to the advanced configuration to ready the catheter for deployment of the next stent. The second collapsed stent is longitudinally spaced apart proximally from the first stent, and the stent stop is configured to radially collapse as the inner tubular member is proximally withdrawn into the outer tubular member through the second collapsed stent, and radially self expand along at least a section thereof at a location proximally adjacent to a proximal end of the second collapsed stent. Thus, the stent stop is slidably positionable to different locations relative to the collapsed stents in the delivery catheter, such that the stent stop initially abuts a proximal end of the first collapsed stent to thereby inhibit proximal movement of the first stent in a first locational configuration, and is configured for being slidably positioned to abut the proximal end of the second collapsed stent to thereby inhibit proximal movement of the second stent in a second locational configuration.

In another aspect of the present invention, the delivery catheter of the invention has only one reversibly collapsible stent stop. In alternative embodiments, an additional reversibly collapsible stent stop is provided at the proximal end of the second (next proximal) stent when said (first) stent stop is in the first locational configuration. Similarly, one or more additional reversibly collapsible stent stops can be provided at the proximal ends of any additional proximally spaced stents in the delivery catheter.

In a method of the invention in which a stent is delivered and deployed in a patient's body lumen, after the delivery catheter outer tubular member is proximally retracted to cause the first stent to radially self expand in the patient's body lumen, the delivery catheter inner tubular member is then proximally withdrawn relative to the outer tubular member and remaining collapsed stents, to position the stent stop at the proximal end of the proximally-next collapsed stent, to ready the catheter for deployment thereof. In one aspect of the invention, the method more specifically includes advancing the stent delivery system to position the first collapsed stent at a desired treatment site in the body lumen with the outer tubular member in the advanced configuration, and deploying the first stent by proximally retracting the outer member relative to the inner tubular member and first stent so that the first stent radially self expands to a deployed configuration in the body lumen with the stent stop in the first locational configuration, such that the stent stop inhibits proximal movement of the first stent, and proximally withdrawing the distal section of the inner tubular member into the outer tubular member in the retracted configuration and thereby transitioning the outer tubular member from the retracted to the advanced configuration, and positioning the stent stop in the second locational configuration by collapsing the stent stop by proximally withdrawing the stent stop (and inner tubular member secured thereto) through the collapsed second stent and radially self expanding the stent stop along at least a section thereof at a location proximally adjacent to the proximal end of the second collapsed stent.

The second stent (and any subsequent stents) can be deployed adjacent to the previously deployed stent(s), or, partially overlapping the previously deployed stent (s) so as to provide continuous scaffolding for a section of the vessel that is longer than one stent, or, fully overlapping the previously deployed stent(s) so as to provide additional scaffolding in the same area of the first stent. Alternatively, the stent delivery system can be repositioned in the body lumen or in a different body lumen prior to deployment of the next stent(s). The stents can have different characteristics from each other, to provide a range of treatment options. For example, the stents can have different maximum deployed outer diameters or lengths. Additionally, in one embodiment, only some of the stents are configured for drug delivery (e.g., drug coated), which facilitates keeping the intervention under the body systemic drug limit. Similarly, some stents can be configured to deliver one drug whereas one or more of the other stents deliver one or more other drugs, which would allow tailoring the drug delivery to various different anatomies or disease states.

Alternatively, the second stent can be a covered stent that can be used in situations when the vessel wall may be accidentally ruptured by a previously deployed stent. In this situation, it is critical to quickly deploy the covered stent to stop the bleeding out of the body vessel. Vessel wall rupture most often occurs after a self-expanding stent has been deployed and a catheter having an expansion member (typically an expandable balloon) is used to further expand the vessel lumen at stent site. An expandable balloon portion could be incorporated into the present invention such that the balloon portion is located distal to the stents which are mounted on the catheter. The balloon portion could be initially utilized to pre-dilate the vessel lumen at the lesion site. A stent can then be deployed into the body lumen. The balloon portion can then be retracted proximally to position the balloon within the deployed stent to allow the balloon to be utilized to further expand the lesion at the stent site. In the event that the vessel wall should rupture, then the covered stent could be deployed within the previously deployed stent. The balloon portion can then be inflated within the covered stent for a period of time which allows the pressure exerted by the balloon to stop any bleeding. The balloon portion can then be deflated and removed from the patient after bleeding has stopped. The use of this balloon portion distal to the pre-mounted stents allows the physician to both pre-dilate the area of stenosis before the stent id deployed and to post-dilate the stenosis after the stent is deployed in the body vessel. Subsequent stents could then be deployed and dilated in multiple sites or to form a continuous scaffolding. This combination of pre-mounted stents and an expandable member, such as an expandable balloon, provide a delivery system which allows for fast and efficient intervention. It should be appreciated that a catheter made in accordance with the present invention having multiple pre-mounted stent also could be used to perform a procedure utilizing a separate catheter having an expandable balloon member. In this procedure, the balloon catheter could be used to pre-dilate several sites or one continuous site, as needed, with the multi-stent catheter then being used to deploy several stents as needed. Thereafter, the balloon catheter could be positioned again to post-dilate the deployed stents.

The delivery catheter provides for accurate deployment of multiple stents at desired treatment sites in the patient's anatomy, due at least in part to the stent stop which is configured to radially collapse and expand as it is slidably positioned within the delivery catheter, and which is radially expanded in a longitudinal gap between a distal collapsed stent ready to be deployed and the proximally-next collapsed stent configured for deploying after the distal stent is deployed. Additionally, the length of the inner tubular member extending beyond the outer tubular member is kept from increasing after each stent deployment by proximally withdrawing the inner tubular member back into the outer tubular member after each stent deployment. As a result, the stent delivery catheter system facilitates quick and easy delivery and deployment of multiple stents to adjoining or different desired locations in the patient's anatomy.

In another aspect of the present invention, a stent delivery catheter can include a pair of stent retainers, each having a portion secured to the elongate shaft member forming the stent delivery catheter and a portion which is slidingly disposed on a portion of the expandable member (for example, an inflatable balloon). Each stent retainer is designed to initially engage an end of the collapsed stent to thereby inhibit proximal and distal movement of the stent from its mounted position on the balloon. The stent retainer can engage the stent by abutting against the end of the stent to prevent longitudinal movement. Alternatively, the stent retainer can engage the stent by having the stent contact portion of the retainer placed over the end of the stent. The other portion of the stent retainer can be fixedly attached to the elongate shaft member forming the catheter or it can be frictionally secured to the shaft member.

In another aspect of the invention, each stent retainer can include at least a pair of cylindrical rings attached together which are adapted to expand with the expandable member as it expands the mounted stent. These rings of the stent retainer move with the expandable member as it partially expands to continue to engage the stent and inhibit proximal movement of the stent form its mounted position.

In another aspect of the invention, one or more stent retainer rings can be used to retain the stent on the stent delivery catheter. Such a stent retainer ring can be disposed on a portion of mounted stent such that the stent retainer ring inhibits both proximal and distal movement of the stent from its mounted position. These retainer rings also could be used on self-expanding stents to prevent the stent from expanding to the expanded position. The retainer ring would have a nominal width and a wall thickness and would include a plurality of tear sections each having a width and/or wall thickness which is less that the nominal width and wall thickness of the remaining portion of the ring. The tear sections of the retainer ring are adapted to be placed between struts of the stent as it is mounted to the expandable member (balloon) and are adapted to break when the balloon is expanded. The retainer ring can be made from a bio-absorbable material which dissolves over time. At least a portion of the retainer ring could be secured to the stent by a bioabsorbable material to ensure that pieces of the ring do not enter the patient's vasculature. The ring can be of a simple cylindrical shape and made from a bioabsorbable material so that as the balloon is expanded the rings simply tear and are wedged between the stent and the vessel wall and remain there until they are dissolved.

These and other advantages of the invention will become more apparent from the following Detailed Description and accompanying exemplary drawings.

### BRIEF DESCRIPTION OF THE DRAWINGS

FIG. 1 is an elevational view, partially in section, of a stent delivery system embodying features of the invention.

FIG. 2 is a transverse cross sectional view of FIG. 1, taken along line 2-2.

FIG. 3 is a transverse cross sectional view of FIG. 1, taken along line 3-3.

FIGS. 4-10 illustrate the system of FIG. 1 in a patient's body lumen during deployment of a first and a second stent.

FIG. 11 is an elevational view, partially in section, of a distal end section of an alternative embodiment of a stent delivery system embodying features of the invention, having a first, second and third reversibly collapsible stent stop illustrated in a patient's vasculature ready for deployment of the first stent.

FIG. 12 illustrates the system of FIG. 11 in a patient's vasculature after deployment of the first stent.

FIG. 13 illustrates the system of FIG. 11 in a patient's vasculature after deployment of the first stent

FIG. 14 is a cross-sectional elevational view of a balloon expandable stent delivery system embodying features of the invention.

FIGS. 15-18 illustrate the system of FIG. 14 in a patient's body lumen.

FIGS. 19-21 are cross-sectional views of outer tubular members which can be used on a multiple stent delivery system.

FIG. 22 is a cross-sectional elevational view of an alternative stent delivery system including an expandable member, such as a balloon, which embodies features of the invention.

FIGS. 23-26 are cross-sectional views showing alternative embodiments of stent stops which could be used with the stent delivery systems disclosed herein.

FIGS. 27A and 27 B are cross-sectional views showing an embodiment of a stent retainer affixed to the distal end portion of a stent delivery catheter.

FIGS. 28A and 28 B are cross-sectional views showing an alternative embodiment of a stent retainer affixed to the distal end portion of a stent delivery catheter.

FIG. 29A is a cross-sectional view showing a stent retainer engaging a stent by forming an abutting shoulder which prevents the stent from moving proximally along the length of the stent delivery catheter.

FIG. 29B is a cross-sectional view showing a stent retainer engaging a stent by overlapping the proximal end of the mounted stent to prevent the stent from moving proximally along the length of the stent delivery catheter.

FIG. 29C is a cross-sectional view showing a stent retainer engaging a stent by forming an abutting shoulder and partially overlapping the mounted stent to prevent the stent from moving proximally along the length of the stent delivery catheter.

FIGS. 30A, 30 B and 30C are cross-sectional views showing an alternative embodiment of a stent retainer affixed to the distal end portion of a stent delivery catheter.

FIG. 31A is a side elevational view showing an embodiment of a stent retainer ring affixed to a stent mounted on the distal end portion of a stent delivery catheter.

FIG. 31B is a cross-sectional view showing the stent retainer ring of FIG. 31A as it affixes a stent to the expandable member of a stent delivery catheter.

FIG. 31C is a side view showing the wall thickness of a portion of the stent retainer ring of FIG. 31A.

FIG. 31D is a front view showing the width of a portion of the stent retainer ring shown in FIG. 31C.

### DETAILED DESCRIPTION OF THE PREFERRED EMBODIMENTS

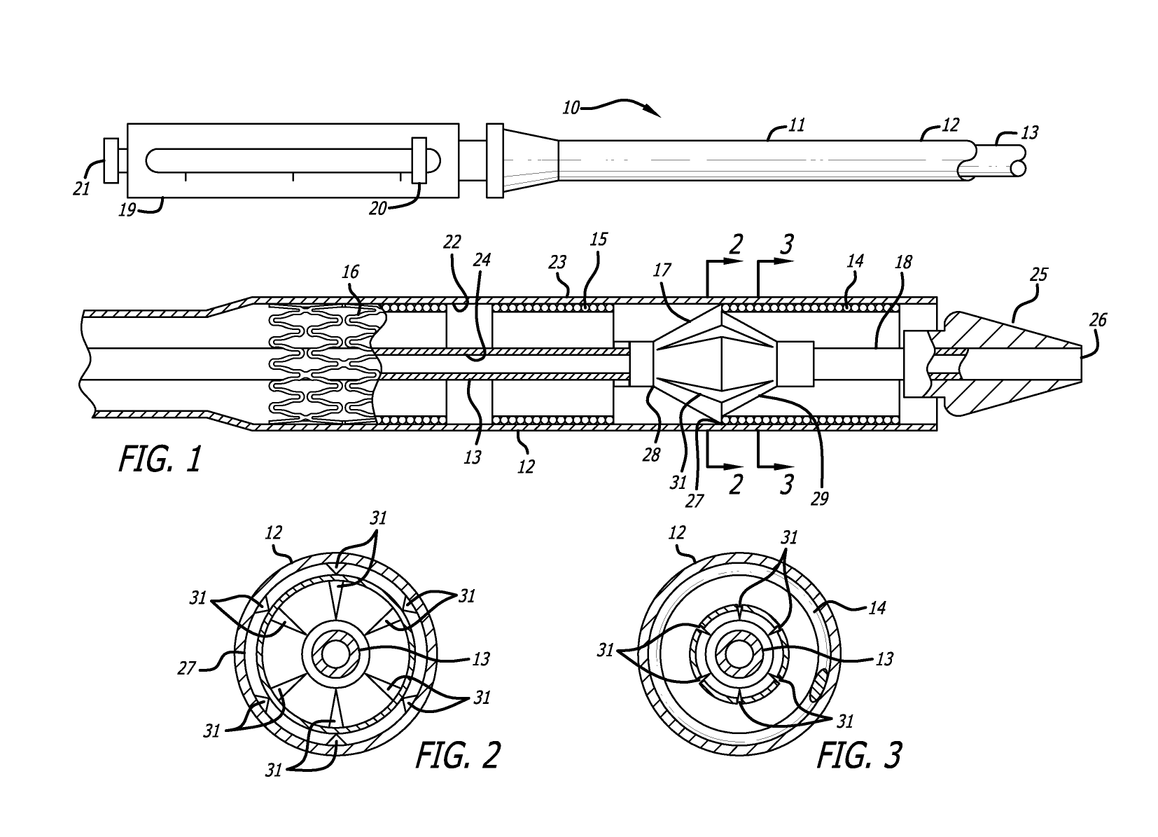

FIG. 1 is an elevational, partially in section, view of a stent delivery system 10 embodying features of the invention, generally comprising a delivery catheter 11 having an outer tubular member 12 and an inner tubular member 13 adapted for axial movement with respect to each other, a first stent 14, a second stent 15, and a third stent 16, each stent being a self expanding stent and in a collapsed configuration within the delivery catheter, and configured to radially self expand from the collapsed configuration to an expanded configuration upon removal of a radially restraining force of the catheter outer tubular member, and a reversibly collapsible stent stop 17 secured to the inner tubular member 13. The outer tubular member has an advanced configuration surrounding a distal section 18 of the inner tubular member, and a proximally retracted configuration, and the inner tubular member is configured for proximally withdrawing the distal section 18 of the inner tubular member 13 into the outer tubular member in the retracted configuration and thereby transitioning the outer tubular member from the retracted to the advanced configuration. The proximal ends of the inner and outer tubular members are connected to a proximal handle 19, typically with the outer tubular member operatively connected to a first mechanism 20 such that operation of the handle first mechanism 20 causes the outer tubular member to move axially as the inner tubular member is held in place during stent deployment, and the inner tubular member operatively connected to a second mechanism (not shown) such that operation thereof causes the inner tubular member to move axially as the outer tubular member is held in place following a stent deployment in order to ready the catheter to deliver the next stent, as discussed in more detail below.

In the illustrated embodiment, the first mechanism 20 is configured to slide proximally to effect proximal retraction of the outer tubular member. A variety of suitable handles can be used with a catheter of the invention, generally having a thumb wheel and/or slide activated mechanism. Generally, slide mechanisms, such as the one shown, may be preferred for coronary applications while handles utilizing a thumb wheel as the actuating mechanism may be preferred for peripheral applications where the stents may be longer in length and a higher delivery force is needed to retract the other tubular member. In peripheral cases, a thumb wheel mechanism can provide a mechanical advantage to overcome the initial delivery force resulting from longer and multiple stents. Fittings such as luer fitting 21 are typically provided at the proximal end of the catheter handle for making a fluid connection to an inner lumen of the catheter (e.g., for flushing out the lumen), and may also provide access for a guide wire into a guide wire lumen of the catheter. The handle typically has lock mechanisms which can be separately engaged to prevent unwanted longitudinal movement of the inner and outer tubular members individually. FIG. 1 illustrates the catheter system 10 with the outer tubular member 12 in the advanced configuration and the stents 14, 15, 16 in the collapsed configuration, and FIGS. 2-3 illustrate transverse cross sections of the catheter system 10 taken along lines 2-2 and 3-3, respectively.

Although illustrated with three stents 14, 15, 16, it should be understood that a stent delivery system of the invention more generally has a first (distal) stent and one or more additional stents longitudinally spaced apart proximally from the first stent and from each other, in a collapsed configuration in the outer tubular member, configured to deploy by radially self expanding from the collapsed configuration to an expanded configuration upon removal of the radially restraining force of the catheter outer tubular member. The stents are configured to deploy in order from the first stent to each successive next proximal one or more additional stents, and the outer tubular member has a first advanced configuration surrounding the distal section 18 of the inner tubular member 13 and the first stent 14, and one or more additional advanced configurations surrounding the distal section 18 of the inner tubular member 13 and each successive next proximal one or more additional stents (e.g., 15, 16) which are in the collapsed configuration in the outer tubular member 12.

The collapsed stents in the delivery catheter are in an annular space 22 between the outer tubular member 12 and the inner tubular member 13, with the outer surface of the stents in contact with the inner surface of outer tubular member radially spaced above the outer surface of the inner tubular member. The outer tubular member 12 is typically formed of multiple tube sections providing tailored performance characteristics along the length of the catheter. For example, in the illustrated embodiment, the outer tubular member has a stent restraining distal tube or distal outer member or sheath 23 which is configured to radially restrain the collapsed stents therein. Its proximal end is bonded to a distal end of a proximally adjacent tube of the outer tubular member, which is typically more flexible than the distally adjacent distal sheath. A lubricious layer or coating (not shown) on the inner surface of the stent restraining tube 23 can be provided to facilitate proximally retracting the outer tubular member during stent deployment. Although not illustrated, an outer-most tubular member may be provided on a proximal end section of the outer tubular member 12 to provide additional stabilization. The inner tubular member 13 may be similarly formed of multiple tubes joined end to end, and/or multiple layer tube(s). Additionally, a stent holder layer can be provided on an outer surface of the sections of the inner tubular member that is surrounded by the collapsed stents 14, 15, 16, which decreases the size of the gap between the inner surface of the collapsed stent and the outer surface of the inner tubular member to improve catheter deliverability.

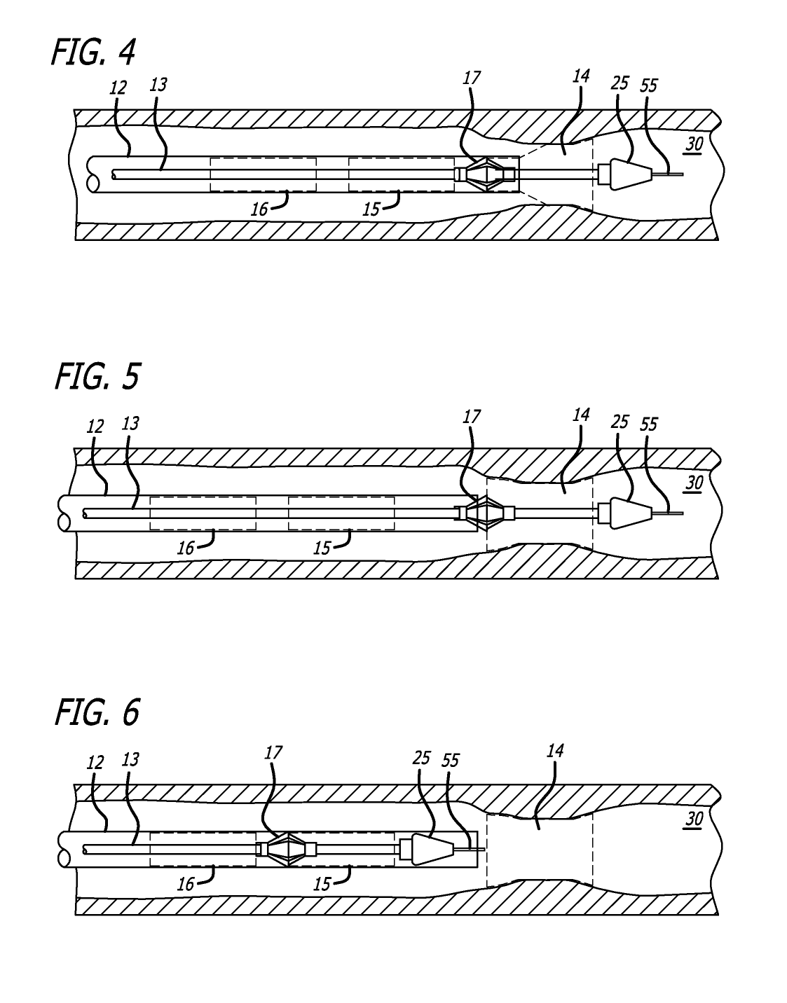

The inner tubular member 13 has a lumen 24 therein. In the illustrated embodiment, the inner tubular member lumen 24 is configured to slidably receive a guide wire (see FIGS. 4-10) used to position the system 10 in the patient's body lumen. In performing a procedure, the guide wire is first introduced into the patient's body and is manipulated until its distal end is just past the lesion site. The distal end of the multi-stent catheter is then fed over the proximal end of the guide wire and into the body. The catheter is continued to be fed distally down the guide wire until its distal end is at the distal end of the lesion site. In the particular embodiment shown in FIGS. 1-10, the guide wire lumen 24 extends the full length of the inner tubular member. However, alternatively, the guide wire lumen 24 can be a short tubular member within at least a distal section of the inner tubular member which provides an alternative embodiment to define the guide wire lumen, particularly in embodiments configured for rapid exchange, having a guide wire proximal port spaced distally from the proximal end of the catheter shaft. Alternatively, in one embodiment, the delivery catheter 11 may have a distal tip wire, configured to facilitate advancing the distal end of the entire system into desired anatomy, secured to the distal end of the delivery catheter such that the system 10 is a fixed wire-type system, and is not configured to slidably advance over a guide wire. Such a system is generally known as a “fixed wire” system which provides the physician with an alternative delivery procedure to the over-the-wire technique disclosed in FIGS. 1-10.

A distal tip member 25 at the distal end of the inner tubular member is configured to preferably reduce trauma to the patient's body lumens as the system 10 is advanced therein. In the illustrated embodiment, the distal member 25 includes a proximal stem fixedly secured to the distal end of the inner tubular member 13, and with a guide wire distal port 26 in a distal end of the distal tip member 25. However, the distal tip member 25 can alternatively be formed as an integral, one-piece extension of the distal end of the catheter shaft. The distal tip member 25 has a radially enlarged (maximum outer diameter) section which in the illustrated embodiment is substantially flush with the distal end of the outer tubular member stent restraining region 23, such that it provides a gradually tapering surface in front of and covering the end of the outer member, to facilitate atraumatically maneuvering the catheter through the patient's tortuous anatomy. Also, the maximum diameter of the tip can have a radiused edge instead of a sharp corner. This will also prevent vessel wall damage, especially when the outer tubular member is moved proximally when deploying a stent and the tip is substantially distal of the distal end of the other tubular member. Also, after the stent is deployed, the tip will be distal of the stent and will need to be pulled through the deployed stent. A radius on the outer edge of this tip 25 will help to prevent the tip 25 from catching behind the distal end of the deployed stent. The maximum outer diameter of the distal tip 25 can be smaller than the inner diameter of the stent restraining region 23 of the outer tubular member 12, or if larger it can be configured to be radially collapsible, to allow the inner tubular member 13 to be proximally withdrawn into the outer tubular member 12 a distance greater than the length of the stent(s), so that the stent stop 17 can thereby reach the proximal end of the next stent to be deployed when the stent has moved proximally with the outer tubular member during the previous stent deployment. This allows the distal tip member to be drawn into the outer tubular member 12 as the inner tubular member 13 is retracted within the outer tubular member 12.

The distal tip member 25 is typically formed of a relatively soft polymeric material having a lower Shore durometer hardness than at least a layer of the inner tubular member 13 proximally adjacent thereto. In one preferred embodiment, the distal tip member 25 is formed of a blend of polymeric material and radiopaque material such that it is radiopaque, although it could be made radiopaque using a variety of suitable methods including being provided with radiopaque material in form of a marker band, to make at least a portion of the distal tip visible under fluoroscopy during use of the system 10. A marker band is a common component used for visualization. Alternatively, a radiopaque tip could be used as well for visualization.

Secured to the inner tubular member 13, the reversibly collapsible stent stop 17 is illustrated in FIG. 1 in a radially expanded configuration, between the first (distal) stent 14 and the proximally-next (second) stent 15. The stent stop 17 extends around the circumference of the inner tubular member 13 and is typically mounted thereto by adhesive, laser welding or similar bonding techniques, depending on the material used to make the stent stop. As will be discussed in greater detail below, the stent 17 can also be rotatably mounted to the inner tubular member 13. The stent stop 17 has a section with a conical shape which has an outer surface tapering proximally from a maximum outer diameter portion 27 to a smaller outer diameter proximal end 28 in the expanded configuration. In the illustrated embodiment of FIG. 1, the stent stop 17 has a distal section 29 having a smaller outer diameter than the larger outer diameter portion 27, and specifically, in the illustrated embodiment, it has a distally tapering outer diameter. The maximum diameter portion 27 of the conical proximal section of the stent stop 17 is configured to reversibly radially expand and collapsed to a smaller outer diameter than the expanded configuration outer diameter. Specifically, as the inner tubular member 13 is proximally withdrawn such that the proximally tapering outer surface of the stent stop 17 contacts the end and inner surface of the proximally adjacent stent (second stent 15), the collapsed stent 15 forces those portions of the stent stop 17 in contact therewith to radially collapse. The distal section 29 of the stent stop typically has a set outer diameter which does not radially expand or collapse on the inner tubular member as it is slid into and out of the collapsed stent.

The particular embodiment of the stent stop 17 of FIG. 1 includes a proximal end 28 which is fixedly disposed on the inner tubular member 13. This prevents the proximal end 28 of the stent stop 17 from moving longitudinally along the inner tubular member 13. The distal end of the stent stop is shown freely movable on the inner tubular member such that it moves as the stent stop 17 is being expanded or collapsed. The spring force that tends to radially expand the stent stop 17 can be generated by both the proximal end and the distal end of the stent stop. In this manner, the distal end of the stent stop could be fixedly attached (not shown) to the inner tubular member in the same manner in which the proximal stop is attached to the inner tubular member. A higher spring force may thus be generated.

The stent stop 17 is preferably made from a material which is self-expanding, such as nickel-titanium (Nitinol) or similar materials. Spring steel could also be used. The stent stop 17 also should be radiopaque, as for example by being formed of a radiopaque metal such as a nickel-titanium loaded with platinum, or similar materials, or a blend of polymeric and radiopaque materials, although other suitable methods of providing the stent stop 17 with radiopacity can alternatively be used including adding a radiopaque marker to the stop or using fittings made from a radiopaque material.

The stent stop 17 also can be formed from a moldable polymer, such as Nylon or polypropylene, although it can alternatively be formed of Nitinol and other self-expanding materials, as mentioned above. In the illustrated embodiment, and particularly when the stent stop is made from a metal or polymer, one or more slots 31 or other voids formed in a wall of stent stop 17 along larger outer diameter portion 27 and distal section 29 make the stent stop radially springy. As can be seen in FIG. 1, the stent stop 17 includes slots 31 which extend from the distal end to at least part way down the slope of the proximal section. These slots help the stent stop to move between its expanded radial position to a more collapsed position. The ability of the stent stop 17 to repeatedly collapse and then re-expand can be provided by using materials with high yield strengths or shapes or an internal spring (FIG. 26) that biases the stent stop 17 to its expanded radial position.

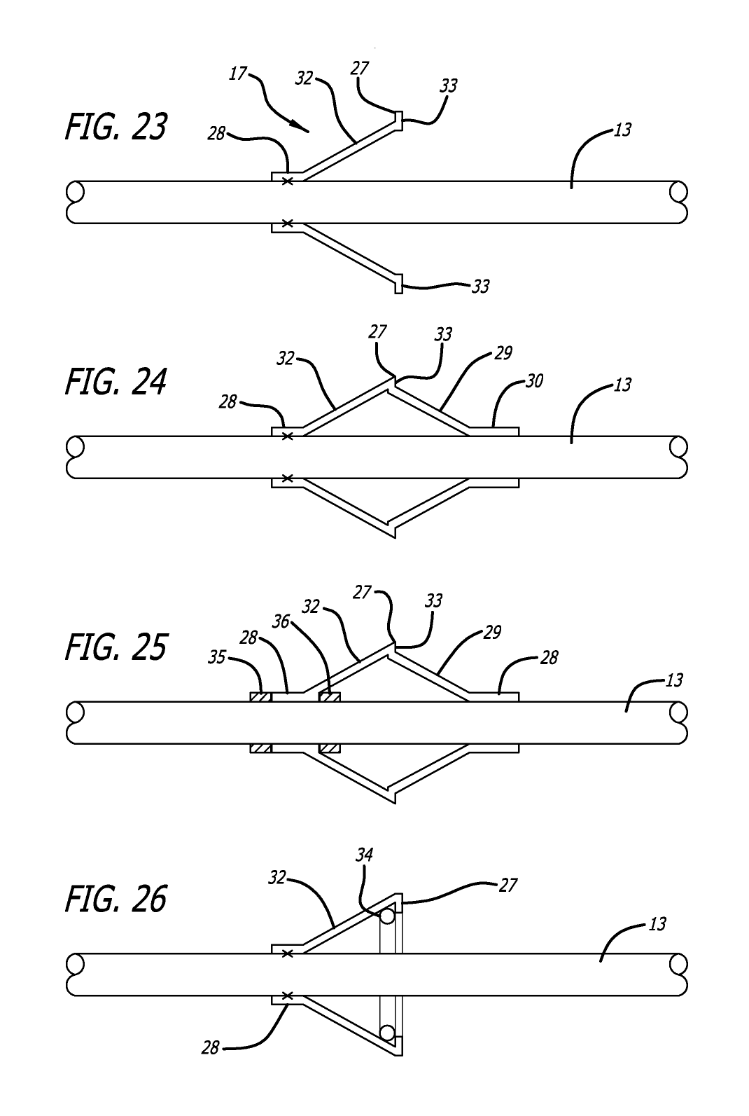

Alternative embodiments of the stent stop are shown in FIGS. 23-26. Referring particularly to FIG. 23, the stent stop 17 is shown without a distal section and merely includes a proximal section 32 with the proximal end 28 attached to the inner tubular member. The proximal section would include an abutting edge 33 along the maximum diameter portion 27 which is designed to abut against the proximal end of the stent (not shown). FIG. 26 shows an alternative embodiment in which a biasing component, such as a circular spring 34, is associated with the stent stop 17 to force the stent stop 17 into the expanded position. A circular spring 34 is just one example of a biasing component which can be used to radially bias the stent stop. FIG. 24 shows an alternative stent stop 17 which has its proximal end 28 fixedly attached to the inner tubular member 13. FIG. 25 shows yet another alternative embodiment in which the proximal end 28 is sandwiched between a pair of fittings 35 and 36 which prevent the stent stop 17 from moving longitudinally along the inner tubular member 13 but allows the stent stop 17 to rotate on the inner tubular member 13. The embodiments of FIGS. 24 and 25 show the distal end 30 of the stent stop 17 being freely movable along inner tubular member 13. Since the distal end 30 is not fixedly attached to the inner tubular member 13, the stent stop 17 of FIG. 25 will be able to rotate on the inner tubular member 13. This particular construction may reduce strain transmitted to the stent via the stent stop 17. Alternatively, the distal end 30 could be rotatable attached to the inner tubular member 13 using a second pair of fittings (not shown) in order to limit longitudinal travel of the distal end 30 along the inner tubular member 13. It should be appreciated that the various embodiments of catheters disclosed herein can be constructed with any of the stent stops disclosed herein. Also, the proximal end 28 and distal end 30 of the stent stop can be made from rings or collars which encircle the outer surface of the inner tubular member 13.

FIGS. 4-10 illustrate the system 10 during the deployment of the stents 14, 15 and 16 in a patient's body lumen 40. In a method of using the system 10 to deliver and deploy at least one of the stents 14, 15, 16, the system 10 is introduced into the patient's anatomy and advanced to a desired treatment site in the body lumen 40 with the outer tubular member 12 in the advanced configuration and the stent stop 17 in a first longitudinal configuration expanded between the first and second stents 14, 15 (see FIG. 1). In the illustrated embodiment, the system 10 is configured to be advanced over a guide wire 55 slidably disposed in the guide wire lumen 24. The radiopaque distal tip 25 and radiopaque stent stop 17 on either side of the first stent 14 are typically visualized under fluoroscopy by the physician to aid in positioning the first stent 14 at the distal end of the desired treatment site. Once in position, the first stent 14 is deployed by proximally retracting the outer tubular member relative to the first stent 14 and inner tubular member 13. The stent stop 17 abuts the proximal end of the stent 14 in the first locational configuration and thereby prevents or inhibits the first stent from moving proximally. Specifically, the maximum outer diameter portion 27 of the stent stop abuts the proximal end face of the stent, and is thus not configured to contact the inner surface of the collapsed stent in the radially expanded configuration. The maximum outer diameter of the stent stop 17 is preferably substantially equal (i.e., within normal manufacturing tolerances) to the outer diameter of the collapsed stents/inner diameter of the outer tubular member to ensure sufficient surface area contact with the abutting end of the stent to prevent proximal movement of the stent. As a result, the first stent 14 does not unintentionally shift position in the outer tubular member stent restraining region 23, which would cause the stent to be implanted in at a different location than the one expected based on the original positioning of the system at the treatment site. Thus, the stent radially expands at the desired location in the body lumen 40. FIG. 4 illustrates the outer tubular member partially retracted and the first stent 14 partially expanded. FIG. 5 illustrates the outer tubular member 12 after it has been fully retracted to the retracted configuration such that the first stent 14 is expanded along its entire length and is thereby deployed at a first treatment site in the body lumen 40.

Following deployment of the first stent 15, the inner tubular member 13 distal section 18, which is now distally spaced from the distal end of the outer tubular member, is proximally withdrawn into the retracted outer tubular member 12. The inner tubular member 13 is withdrawn a sufficient distance to transition the outer tubular member 12 from the retracted to the advanced configuration (i.e., so that the outer tubular member surrounds the distal section 18 of the inner tubular member 13. Additionally, the stent stop 17 is thereby positioned in the second locational configuration between the second and third stents 15, 16. FIG. 6 illustrates the inner tubular member proximally withdrawn such that the stent stop 17 is in the second locational configuration. As discussed above, the stent stop 17 is moved from the first locational configuration at the distal end of the second stent 15 to the second locational configuration at the proximal end of the second stent 15 by collapsing the stent stop as the stent stop is proximally retracted through the collapsed second stent 15. The self-expanding stent stop 17 can then be positioned at a location proximally adjacent to the proximal end of the second collapsed stent 15. Due to the fact that the second and third stents 15 and 16 remain in contact with the outer tubular member 12, these stents 15 and 16 will move with the outer tubular member 12. Also, as previously mentioned above, the distal tip member 25 will be retracted within the outer tubular member 12 as the inner tubular member 13 is retracted into the outer member 12. This allows the stent stop 17 to be moved proximal to the location of the second stent 15. As is shown in FIG. 6, the distal tip member 25 is retracted into the outer tubular member 12.

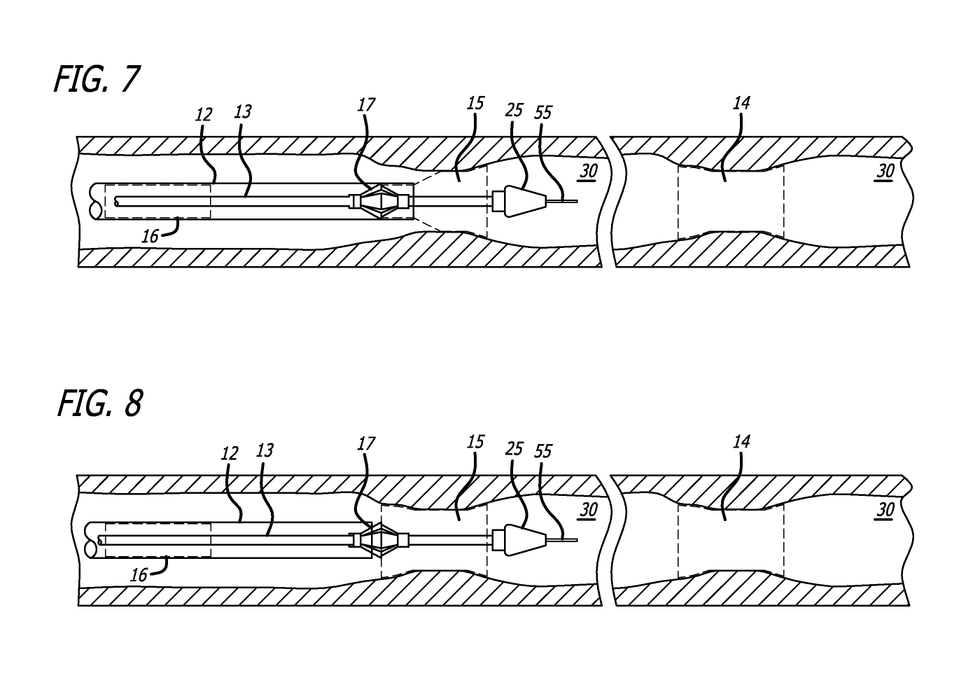

With the outer tubular member 12 in the advanced configuration surrounding the distal section 18 of the inner tubular member and the second stent 15 therearound, the second stent 15 can be deployed either with or without repositioning the stent delivery system 10 in the patient after deployment of the first stent 14. If the system is not repositioned or is only slightly moved in the patient's anatomy, the second stent 15 can be deployed at a desired site adjacent to the first expanded stent 14, with the ends of the expanded stents 14, 15 overlapped (as is typically done for lesions that are longer than a single stent), touching or somewhat spaced apart. Alternatively, the system can be advanced or retracted to a desired treatment site in the body lumen 40 or in a different body lumen, to deploy the second stent 15 remotely from the first expanded stent 14. As before, and as is shown in FIG. 7, the second stent 15 is deployed by proximally retracting the outer tubular member 12 relative to the inner tubular member 13 and second stent 15, with the stent stop 17 in the second locational configuration, such that the stent stop 17 inhibits proximal movement of the second stent 15. FIG. 8 shows second stent 15 fully deployed in the patient's vasculature.

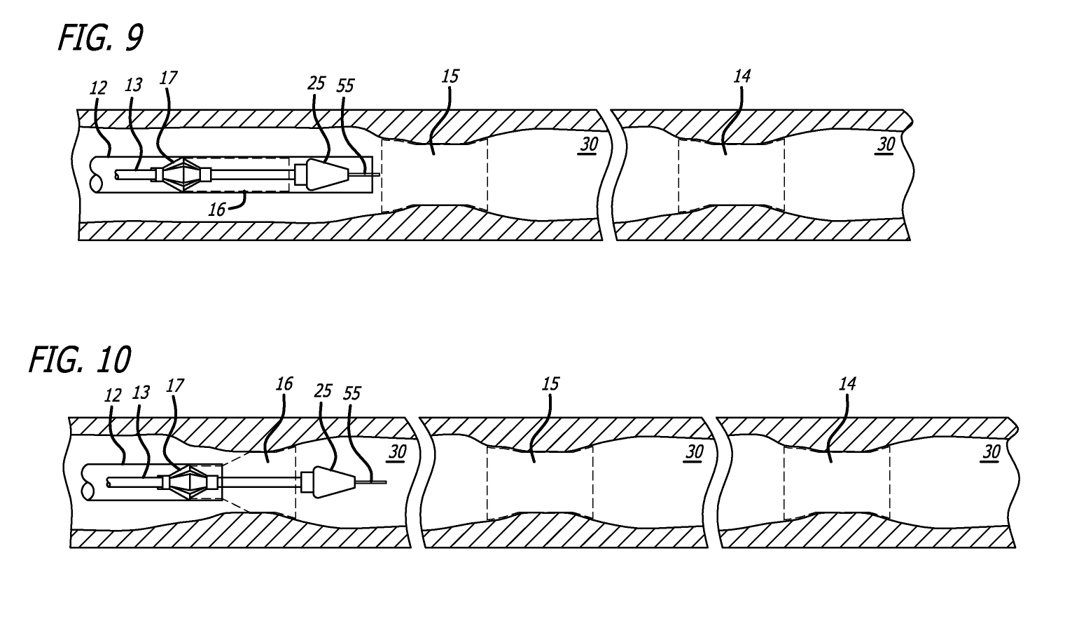

Following deployment of the second stent 15, the inner tubular member 13 can be again proximally withdrawn as before. FIGS. 9 and 10 illustrate the system 10 after the deployment of the second stent 15 at some location in the body lumen 40 remote from the first stent 14, with the inner tubular member 13 proximally withdrawn into the outer tubular member 12, such that the outer tubular member 12 is in the advanced configuration surrounding the distal section 18 of the inner tubular member 13 and the third stent 16, and the stent stop 17 is in a third locational configuration radially self expanded (after collapsing during proximal withdrawing through the third collapsed stent) along at least a section thereof at a location proximally adjacent to the proximal end of the third collapsed stent 16. Again, it should be appreciated that the distal tip member 25 of the inner tubular member 13 will be drawn back into the outer tubular member 12 to allow the stent stop 17 to be placed proximal to the third stent 16. With the outer tubular member 12 in the advanced configuration, the system 10 can again be repositioned, or removed from the patient's anatomy at the end of the procedure.

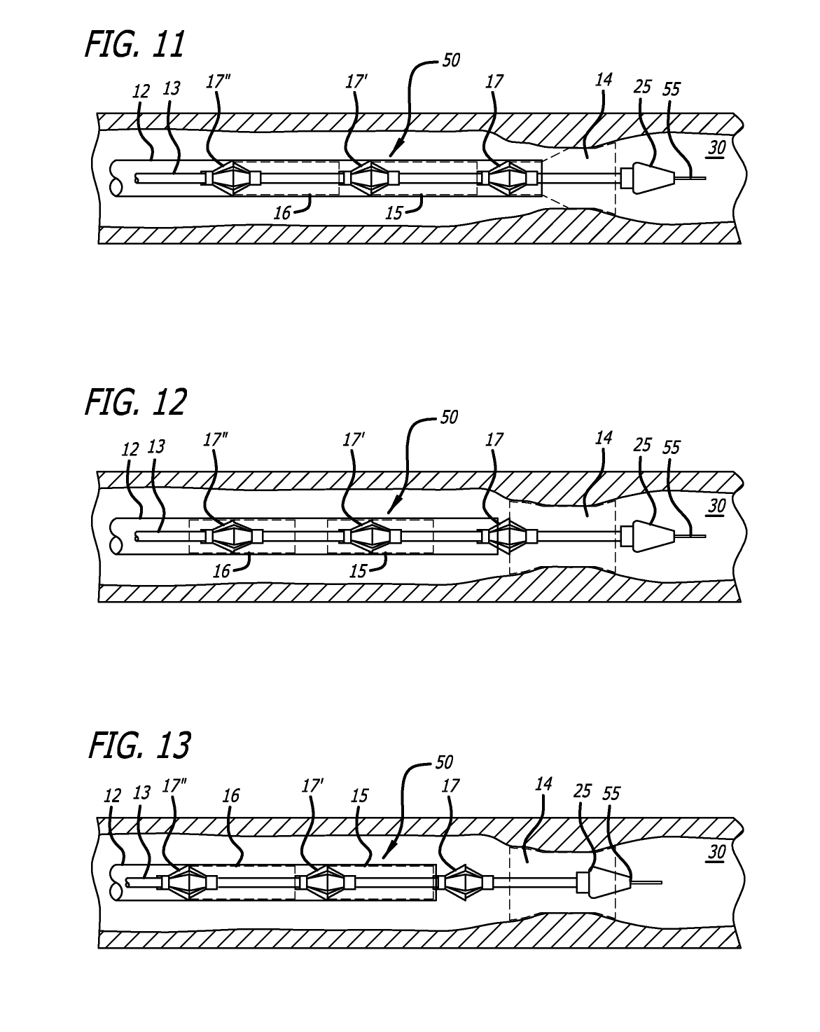

In the embodiment illustrated in FIG. 1, the delivery catheter 11 has a single collapsible stent stop 17. FIGS. 11-13 illustrate the distal end section of an alternative embodiment of a stent delivery system 50 otherwise the same as system 10 but having multiple reversibly collapsible stent stops 17 in accordance with the invention. Each stent stop 17, 17′, 17″ is illustrated in the radially expanded configuration at the proximal end of each collapsed stent 14, 15, 16, respectively. The stent stops 17′ and 17″ at the proximal ends of the second and third stents 15, 16, respectively, prevent or inhibit any unintended proximal movement of the stents 15, 16 during deployment of the first stent 14 (and of stent 16 during deployment of the second stent 15). The additional stent stops 17′ and 17″ are otherwise identical to stent stop 17. FIG. 11 illustrates the system 50 during deployment of the first stent 14, with the outer tubular member 12 only partially retracted. FIG. 12 illustrates the system 50 after deployment of the first stent 15 caused by proximal retracting the outer tubular member 12 relative to the inner tubular member 13. The outer tubular member 12 can then be further retracted, as shown in FIG. 13, to ready the catheter 11 for deployment of the second stent 15. The outer tubular member 12 will then be further withdrawn or retracted to allow deployment of the second stent 15. Prior to deployment of the second stent 15, the catheter can be maneuvered to the particular area where the second stent 15 is to be deployed. The system 10 can be likewise used to deploy the third stent 16.

In contrast, in the embodiment of FIG. 1, the proximal ends of the stents 15, 16 do not abut stent stops 17 during deployment of the first stent 14. A pusher (not shown), such as a tube slidably disposed on the inner tubular member 13 proximal to the proximal-most stent in the annular space 22 between the inner and outer tubular members, can be used to push a collapsed stent distally into position in the outer tubular member 12. Such a pusher can be utilized for a catheter which deploys only two stents. Once the first stent is deployed, the second stent remains positioned back in contact with the outer tubular member

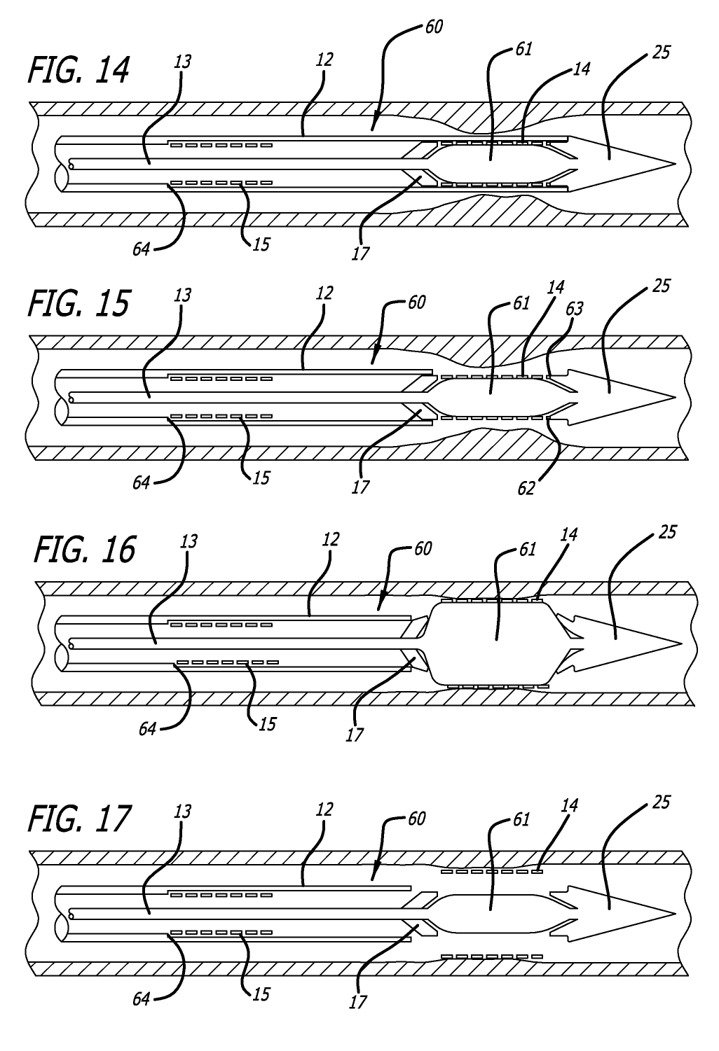

An alternative embodiment of a multiple stent delivery catheter 60 is shown in FIGS. 14-18. This particular stent delivery catheter 60 utilizes an expandable member, such as a balloon 61, which can be utilized to radially expand the multiple stents which are located along the length of the catheter. As can be seen in FIGS. 14-18, two or more stents 14 and 15 can be located on the catheter and can be deployed somewhat in the manner previously described. For example, the stents can either be self-expanding or balloon expandable, or a combination of both. In this regard, the balloon expandable stents would be expanded to the point position within the patient's vasculature by utilizing the balloon for expansion purposes. One of the advantages of utilizing the multiple stent delivery catheter of FIGS. 14-18 includes the elimination of the need to crimp the stents onto the balloon portion 61 of the catheter. The elimination of the need to mechanically crimp the stents onto the catheter can have multiple applications. For example, deployment of stents which have drug, polymer, live cells, membranes or any coatings that may be delicate and could be damaged by the crimping process can now be deployed without risk of possible damage caused by the crimping.

Additionally, stents, such as bio-absorbable stents, are usually susceptible to fracture or radial force degradation from fluctuation of the strain resulting from the crimp. The elimination of the need to crimp such a bio-absorbable stent to the catheter would ultimately help in the final deployment since the bio-absorbable stent would only be expanded from a single application of force caused by the balloon on such a stent. Accordingly, the elimination of the crimping of the bio absorbable stent to the delivery catheter will help to prevent fracture and other deformations which can result from a crimping process.

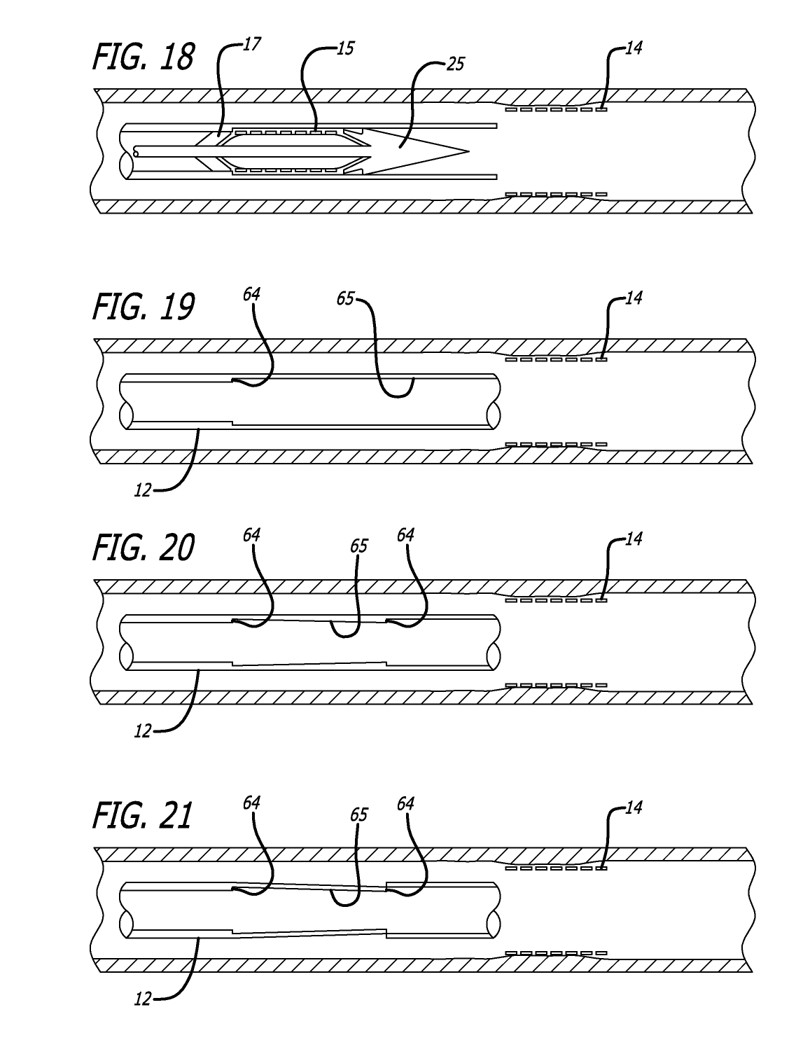

Referring initially to FIG. 14, the balloon delivery catheter 60 is shown including a stent stop 17 which abuts the proximal end of the first stent 14. The distal end 62 of the stent 14 is adapted to abut a proximal edge 63 of the distal tip member 25 of the catheter so that the stent 14 will be longitudinally captured between the distal tip member 25 and the collapsible stent stop 17. FIG. 15 shows the outer tubular member 12 being moved proximally to expose the stent 14 and allow the balloon 61 to expand the stent 14 as is shown in FIG. 13. Once the stent 14 has been deployed in the patient's vasculature, the balloon 61 can be deflated as is shown in FIG. 16. The catheter can then be repositioned to a new deployment site so that the second stent 15, which is mounted on the catheter, can be placed in the desired location for deployment. The inner tubular member 12 of the catheter can be moved proximately until the stent stop 17 is placed proximal to the second stent 15, as is shown in FIG. 18. The outer tubular member 12 may include an internal stent stop 64 which is formed on the inner surface 65 of the outer tubular member 12. This internal stent stop 64 prevents the second loose fitting non-crimped balloon expandable stent 15 from being moved proximally as the inner tubular member 13 and stent stop 17 are moved. It should be noted that the soft distal tip member 25 of the catheter also moves within the outer tubular member 12 in order for the stent stop 17 to fully expand proximal to the second stent 15. Alternatively, the inner tubular member can be moved proximally to capture the second stent and then moved distally so that the second stent is now in the location longitudinally where the first stent was located, as is shown in FIG. 14. The catheter can then be moved to the next deployment site to allow the second stent to be deployed as well.

FIGS. 19-21 show various alternative components which can be utilized as an internal stent stop which is formed directly on the inner surface 65 of the outer tubular member 12. For instance, as is shown in 19, the internal stent stop 64 is molded into the outer member 12 to create a simple abutting shoulder which is used to prevent the stent from moving proximally past the shoulder region. FIG. 20 has multiple internal stent stops 64 molded in the outer member 65 which are suitable for use with multiple loose fitting balloon expandable stents. Lastly, FIG. 21 shows multiple internal stent stops 64 which are offset from the inner surface 64 of the outer tubular member 12.

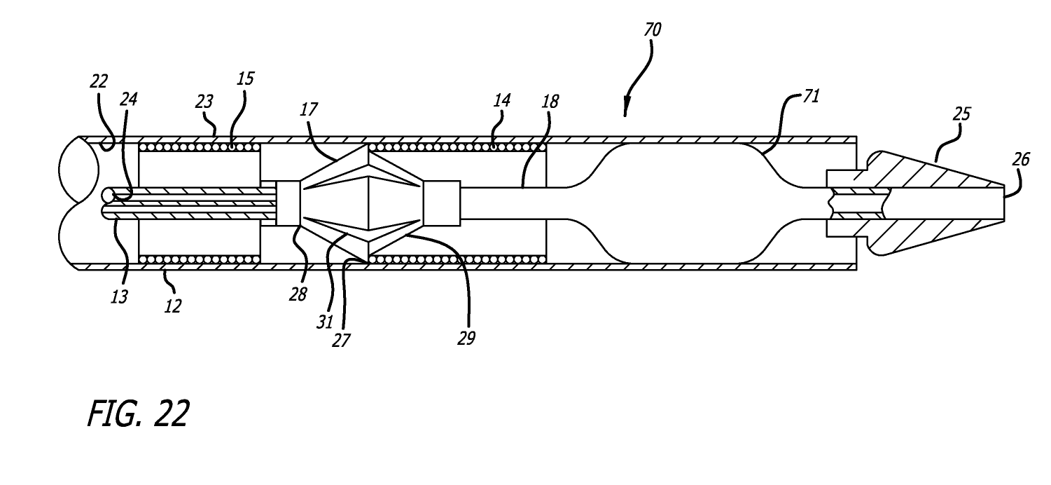

FIG. 22 shows an alternative delivery catheter 70 which includes an expandable member, such as an inflatable balloon 71, which can be used to pre-dilate the artery or to further expand the stent once it has been deployed in the body vessel. This particular embodiment allows the physician to utilize an expandable balloon in conjunction with the stent stops described above. It should be appreciated that FIG. 22 shows just one type of an expandable member which can be utilized with the various stent stops of the present invention.

The second stent (and any subsequent stents) can be deployed adjacent to the previously deployed stent(s), or, partially overlapping the previously deployed stent (s) so as to provide continuous scaffolding for a section of the vessel that is longer than one stent, or, fully overlapping the previously deployed stent(s) so as to provide additional scaffolding in the same area of the first stent. Alternatively, the stent delivery system can be repositioned in the body lumen or in a different body lumen prior to deployment of the next stent(s).

Alternatively, the second stent can be a covered stent that can be used in situations when the vessel wall may be accidentally ruptured by a previously deployed stent. In this situation, it is critical to quickly deploy the covered stent to stop the bleeding into the body vessel. Vessel wall rupture most often occurs after a self-expanding stent has been deployed and a catheter having an expansion member (typically an expandable balloon) is used to further expand the vessel lumen at stent site. An expandable balloon portion could be incorporated into the present invention such that the balloon portion is located distal to the stents which are mounted on the catheter. The balloon portion could be initially utilized to pre-dilate the vessel lumen at the lesion site. A stent can then be deployed into the body lumen. The balloon portion can then be retracted proximally to position the balloon within the deployed stent to allow the balloon to be utilized to further expand the lesion at the stent site. In the event that the vessel wall should rupture, then the covered stent could be deployed within the previously deployed stent. The balloon portion can then be inflated within the covered stent for a period of time which allows the pressure exerted by the balloon to stop any bleeding. The balloon portion can then be deflated and removed from the patient after bleeding has stopped. The use of this balloon portion distal to the pre-mounted stents allows the physician to both pre-dilate the area of stenosis before the stent is deployed and to post-dilate the stenosis after the stent is deployed in the body vessel. Subsequent stents could then be deployed and dilated in multiple sites or to form a continuous scaffolding. This combination of pre-mounted stents and an expandable member, such as an expandable balloon, provide a delivery system which allows for fast and efficient intervention. It should be appreciated that a catheter made in accordance with the present invention having multiple pre-mounted stents also could be used to perform a procedure utilizing a separate catheter having an expandable balloon member. In this procedure, the balloon catheter could be used to pre-dilate several sites or one continuous site, as needed, with the multi-stent catheter then being used to deploy several stents as needed. Thereafter, the balloon catheter could be positioned again to post-dilate the deployed stents.

In another embodiment, the stent stop 17 can be placed closer to the distal tip member 25 up to even contacting the tip (provided the tip is soft so it can retract inside the stent to be delivered, or it is of a smaller diameter than the collapsed stent or made as part of the tip so in this way the tip is a combination tip and stent stop). A method of use would include the following: 1) track the delivery catheter to stent deployment site, 2) retract the inner tubular member until the stent stop is proximal of the first stent and 3) if desired, multiple stents can be deployed by moving the stent stop back past the multiple stents and pushing them out one after the other. The advantage of this system, whether deploying one stent at a time or multiple stents at a time, is that the distal tip member does not extent significantly distal of the outer tubular member as the outer tubular member is proximally retracted when deploying a stent. The advantage of this system is that the possibility that the distal tip member can get caught in a deployed stent is virtually eliminated. Also the tip is not extended distally in the vessel. This is significant in anatomy where distal vessels are small, delicate or torturous. Examples of this type of anatomy are renal and cerebral arteries.

Referring now to FIGS. 27A and 27B, in another aspect of the present invention, a stent delivery catheter 80 can be manufactured to include a pair of stent retainers 82 each having a mounting portion 84 secured to the elongate shaft member 86 forming the stent delivery catheter 80 and an engaging portion 88 which is slidingly disposed on a portion 90 of the expandable member 92 (for example, an inflatable balloon). Each stent retainer 82 is designed to initially engage an end 94 of the collapsed balloon expandable stent 96 that is loose fitting, partially crimped or crimped on the balloon to thereby inhibit movement of the stent 96 from its mounted position on the balloon 92. The stent retainer 82 located on the distal most end of the balloon can be separate from the tip or can be formed as a part of the tip as shown in FIGS. 14 through 18. The stent retainer 82 can engage the stent 96 by abutting against the end 94 of the stent to prevent longitudinal movement. The mounting end 84 of the stent retainer 82 is fixedly attached to the elongate shaft member 86 by suitable means such as adhesive, a heat weld, laser bonding and other attachment techniques well known in the art. The emerging portion 88 of the stent retainer 82 is not fixedly secured to the balloon 92, but rather, is allowed to slide over a portion 90 of the balloon 92. This allows the stent retainer 82 to expand with the balloon, as is shown in FIG. 27B, and not bind the expansion of the balloon. The engaging portion 88 is typically sized so that it will not expand to the full diameter of the vessel into which the intervention is being performed. This is to insure that the engaging portion 88 does not get pinched between the expanding balloon and the vessel wall. The retainer 82 would expand with the balloon until it reached its maximum diameter and then be pushed longitudinally away from the balloon, but still in contact with the balloon, as the balloon continued to expand. The distal most end 98 of the stent retainer 82 is adapted to create a raised shoulder against which the proximal end 94 of the stent 96 abuts. This structure helps to prevent the stent 96 from moving longitudinally along the catheter when, for example, the catheter is being advanced through the patient's vasculature. Torturous anatomy can cause crimped stents to loosen on a balloon. By having both proximal and distal stent retainers will prevent the stent from slipping off the balloon. This stent retainer 82 could also be use when a retractable sheath is placed over the stent during delivery.

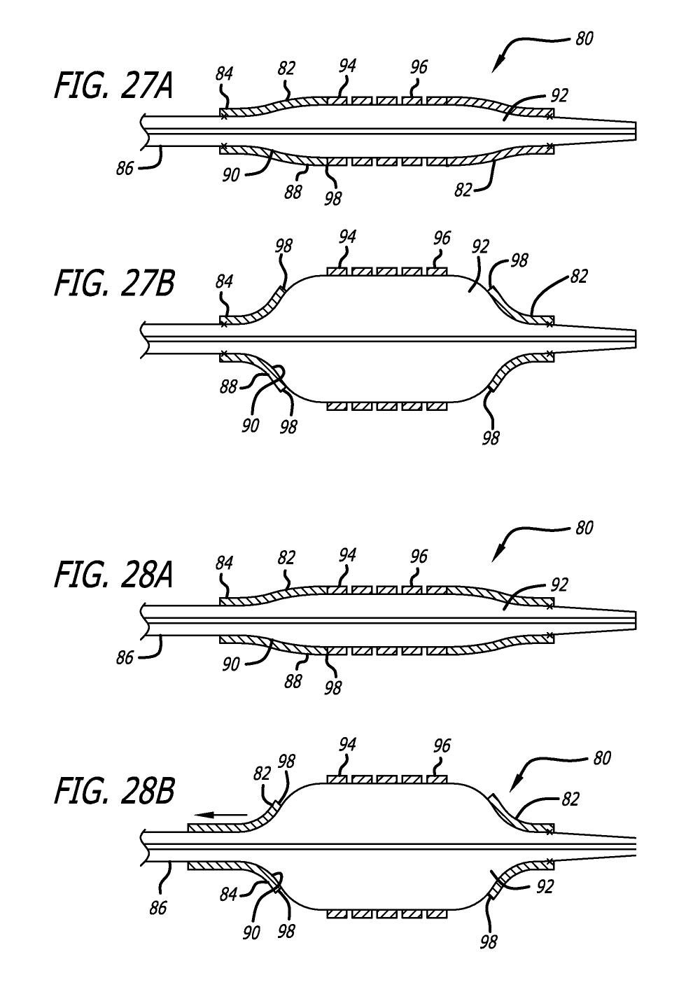

Referring now to FIGS. 28A and 28B, the stent retainer 82 can be frictionally secured to the elongate shaft member 86. FIGS. 28A and 28B show the retainer 82 placed over the shaft member 86. The mounting portion 84 can be made from a material which frictionally engages the surface of the shaft member 86 to maintain the retainer 82 positioned on the catheter during stent delivery. The remaining portion of the retainer 82 which comes in contact with the balloon 92 could be made from an alternative material which allows that portion to slide freely along the balloon portion of the catheter. Alternatively, the contact surface of the retainer 82 could be coated with a material of fluids which decreases sliding friction between that portion of the stent retainer and the balloon. The use of friction to secure the retainer to the catheter provides the assembler of the catheter the ability to move and realign the retainer in relation to the stent in order to achieve proper placement of components. In the embodiment of FIGS. 27A and 27B, since the mounting portion of the retainer 82 is fixed to the tubular member 86, the assembler would not be able to move and realign the retainer as may be needed.

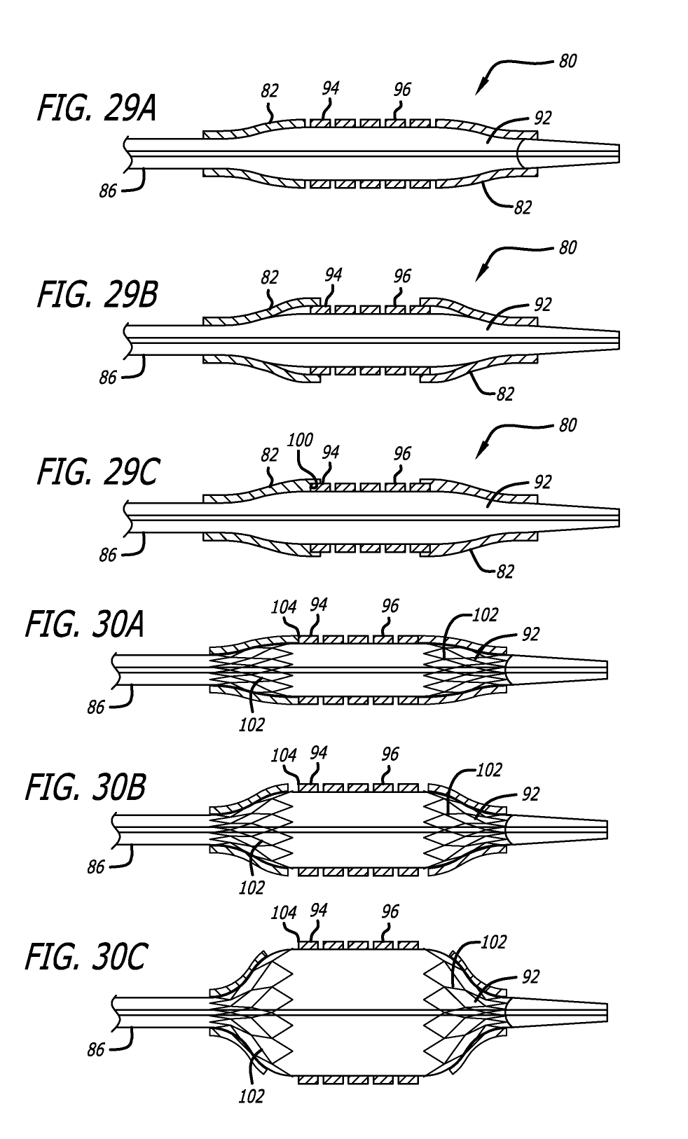

Referring now to FIGS. 29A-29C, various embodiments of the end 98 of retainer 82 are disclosed. In FIG. 29A, the end 98 creates the shoulder which abuts against the end 94 of the stent 96. In FIG. 29B, the retainer 82 engages the stent by having the end 98 overlap the end 94 of the stent 96 keeping the stent in place. In this case, the end 98 would be radiused so as not to cause vessel trauma during delivery. In FIG. 29C, the distal end 98 of the retainer includes an abutting shoulder 100 and a portion which overlaps the stent 96. This particular embodiment provides a strong structure for preventing stent movement along the catheter. While the stent retainer 82 is shown formed as a tube-like sleeve in the disclosed embodiments, it should be appreciated that other shapes and structures could be used to create the retainer. The retainer 82 should also be made from a material which is capable of stretching to allow the retainer 82 to expand with the balloon as it is expanded. Otherwise, the retainer could prevent the end of the balloon from fully inflating to its proper diameter. However in the case of the friction fit of FIGS. 28 A and B, it could be a molded polymer of formed metal part. On balloon expansion, it would be pushed by the balloon sideways away from the balloon as friction was overcome to allow the balloon to expand.

In another embodiment of the invention, depicted in FIGS. 30A-30C, the stent retainer 82 is made from at least a pair of cylindrical rings 102 attached together which are adapted to expand with the balloon 92 as it expands the mounted stent 96. These rings 102 move with the balloon 92 as it partially expands (see FIG. 30B) to continue to engage the stent 96 and inhibit proximal movement of the stent from its mounted position. These rings 102 are somewhat like an expandable stent as they are able to expand radially with the inflating balloon 92. The rings 102 include a shoulder region 104 against which the proximal end 94 of the stent abuts. When the balloon 92 is fully expanded, as is shown in FIG. 30C, the rings 102 are also expanded to maintain the shoulder against which the stent abuts. The fully expanded diameter of the rings 102 could be smaller than the diameter of the fully expanded balloon to allow the rings 102 to fully expand before the balloon fully expands. A fully expanded stent-like retainer could cause unnecessary vessel trama by being pushed against the vessel wall by the fully expanded balloon.

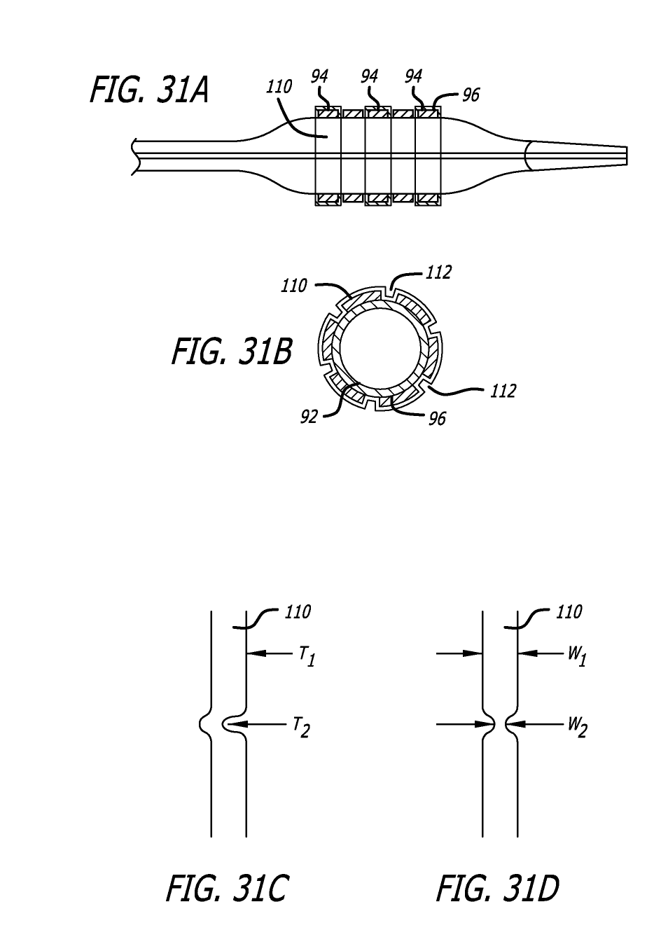

In another aspect of the invention, depicted in FIGS. 31A-31D, the stent delivery catheter 80 can manufactured with one or more stent retainer rings 110 which can be used to retain the stent 96 on the stent delivery catheter. Such a stent retainer ring 110 can be disposed on a portion of mounted stent 96 such that the stent retainer ring 110 inhibits proximal movement of the stent from its mounted position. This system can apply to a balloon expandable stent and a self expanding stent on a balloon. In the latter case the retainer 110 also restricts radial expansion of the stent. The retainer ring 110 has a particular width W₁ and a wall thickness T₁, as is shown in FIGS. 31C and 31D, along with a plurality of tear sections 112 which have a width W₂ and wall thickness T₂, which is less that the width W₁ and the wall thickness T₁ of the remaining portion of the ring. These tear sections 112 of the retainer ring 110 can be adapted to be placed between struts of the stent (see FIG. 31B) as the stent 96 is mounted to the balloon. These tear sections 112 are adapted to break when the balloon 92 begins to expand. The retainer ring 110 can be made from a biodegradable material, such as Poly Lactic Acid which readily dissolves over time in the patient's vasculature. At least a portion of the retainer ring could be secured to the stent by utilizing a bio-absorbable material, such as Poly Lactic Acid, which maintains the pieces of the retainer ring 110 fully attached to the stent after deployment in the patient's vasculature. The retainer rings 110 can be crimped onto the mounted stent or preformed prior to placement on the stent.

The various stents in the delivery catheter can be all the same, or have one or more different characteristics. The stents can have different characteristics from each other, to provide a range of treatment options. For example, the stents can have different maximum deployed outer diameters or lengths. Additionally, in one embodiment, only some of the stents are configured for drug delivery (e.g., drug coated), which facilitates keeping the intervention under the body systemic drug limit. Similarly, some stents can be configured to deliver one drug whereas one or more of the other stents deliver one or more other drugs, which would allow tailoring the drug delivery to various different anatomies or disease states. In one embodiment, two or more of the stents have different lengths. Similarly, two or more of the stents can have different outer diameters. Preferably, the handle 19 would be marked with the stent sizes so that there would be direct communication to the physician of the size of the next stent to be deployed. Also the stent stop could be made from a radiopaque material so that the doctor could visually see when the stent stop was correctly positioned behind the stent being deployed. Additionally, other characteristics such as whether or not the stent has a drug or other agent coated or otherwise applied thereto, the amount of the drug, and the nature of the drugs delivered by the multiple stents can all vary amongst the different stents. By way of example, the maximum expanded diameter of stents useful in a system of the invention typically ranges from of about 2 to about 10 mm, and the maximum expanded length ranges from about 10 to about 200 mm. This size range covers peripheral and coronary applications and self expanding and balloon expandable metal and bioabsorbable stents.

In a presently preferred embodiment, a catheter system of the invention is configured for delivering and deploying one or more of the stents in the patient's superficial femoral and iliac arteries, although it could be configured for use in a variety of body lumens, including other peripheral and coronary vessels and non-vascular body lumens.

The stent retainers disclosed in FIGS. 27-29 can be made from polymeric materials such as _Pebax, Pebax loaded with a radiopaque material. The embodiment of FIGS. 31A to 31D could also be made from peek, nylon, high durometer Pebax, polycarbonate, stainless steel. The rings 102 disclosed in the embodiment of FIGS. 30A-30C can be made from polymeric material, Nickel-titanium alloys and other suitable materials which will allow the rings to expand with the balloon.

The catheter components, such as the inner and outer tubular members, can be formed of materials found useful in catheter construction. For example, the polymeric tubular members can be formed of materials such as polyamides, polyamide copolymers (e.g., polyether block amide), polyolefins (e.g., polyethylene), polyurethanes, polyesters, and the like. Generally speaking, the more proximal portions of the catheter inner and outer tubular members will be stiffer than the distal portions, to provide the catheter sufficient pushability, and the catheter distal section is configured to provide flexibility and trackability to advance through the patient's vascular system by tracking on a wire in the lumen. The distal sheath 23 that covers the stent before it is deployed, and particularly for self-expanding stents that apply an outward force in the radial direction on the distal sheath, needs to have a high resistance to radial expansion. This is typically achieved through thin rigid materials such as polyimide or a more flexible braded material where a metal bead is encapsulated by nylon or other suitable polymer.

A multilayered balloon could be used with the other components of the present invention. Such a multilayered balloon could include a first layer and at least a second layer, and could have noncompliant limited radial expansion beyond the nominal diameter of the balloon. By selecting the polymeric materials forming the balloon layers, and arranging and radially expanding the multiple layers of the balloon, one can create a balloon that has improved low compliance, preferably in combination with high flexibility and softness. Such a multilayered balloon can be formed in whole or in part of coextruded polymeric tubular layers. A multilayered balloon is typically formed by conventional blow-molding in which a multilayered polymeric tube is radially expanded within a balloon mold. The resulting multilayered balloon has an inflated shape which corresponds to the inner surface of the mold and which has a diameter about equal to the inner diameter of the balloon mold, commonly referred to as the balloon's nominal working diameter. The nominal pressure is the inflation pressure required to fill the balloon to the nominal working diameter. The balloon expands a very small amount (i.e., noncompliantly) at pressures above the nominal pressure. As a result, the balloon minimizes injury to a patient's blood vessel, which can otherwise occur if the balloon continues to expand a substantial uncontrolled amount at increasing inflation pressures above nominal.

The blow-up-ratio (BUR) of a balloon formed from a polymer tube should be understood to refer to the ratio of the outer diameter of the blown balloon expanded within the mold (i.e., the mold inner diameter) to the inner diameter of the polymer tube prior to being expanded in the mold. Each individual layer of a multilayered balloon similarly has its own BUR based on the ratio of the inner diameter of the mold and the inner diameter (prior to expansion in the mold) of the layer of the polymeric tube. For a given balloon wall thickness, the rupture strength generally increases and the radial compliance decreases as the balloon BUR increases. For standard pressure driven blow molding of catheter balloons, typical BURs range from about 4.5 to about 8.0 depending on the material and the product application. Specifically, a multilayered balloon can be made with polymeric materials that can be expanded to higher BURs as the inner layer(s) of the balloon, while lower BUR materials are the outer layer(s) of the balloon. The balloon can have a first layer of a first polymeric material and a second layer of a second polymeric material which has a lower Shore durometer hardness than the first polymeric material and which can be expanded during balloon blowing to a higher BUR (without rupturing or tearing) than the higher Shore durometer hardness material of the first layer, and the second layer is an inner layer relative to the first layer. For example, the multilayered balloon inner layer can be formed of a polyether block amide (PEBA) material (e.g., commercially available as PEBAX®) having a Shore durometer hardness of about 60-70D while the outer layer is formed of a PEBA material having a higher Shore durometer hardness of about 70-72D. However, a variety of suitable materials can be used including materials which are of the same material classification/family, or different classes of materials. The multilayered balloon generally includes two or more layers (i.e., layers formed of materials which differ in some respect such as different Shore durometer hardnesses), although it typically does not have more than about five layers.

For example, a suitable multilayered balloon would include a first (outer) layer of a first durometer, and one or more inner layer(s) of successively lower durometers (i.e., increasingly softer materials), has a lower compliance than a balloon having about the same wall thickness but formed of 100% of the highest durometer material (i.e., the material forming the outer-most layer of the balloon). Compared to a balloon formed of 100% of the highest durometer material, a multilayered balloon has effectively replaced a part of the balloon wall thickness with the layer(s) of lower durometer (softer) material(s), which would typically be expected to increase the compliance. While not wishing to be bound by theory, it is believed that the balloon provides the noncompliant behavior through the specific combination of highly oriented layers of the balloon, and particularly by maximizing the orientation of the inner layer(s) of the balloon. The inner layer orientation significantly affects compliance of the balloon. By selecting and arranging different materials that can be blown to different BURs in accordance with the invention, the balloon has layers with successively increasing BURs from the outer to the inner layer(s), such that the BUR of each layer is preferably maximized and the inner layer(s) have particularly high BURs. The layers of the balloon are therefore optimized for compliance purposes. Although additional layers may be added to the balloon, to, for example, increase the total wall thickness to a desired value, the arrangement of the basic layers in accordance with the invention cannot be varied without resulting in a higher compliance balloon.

Another suitable multilayered balloon would include a first (outer) layer of a first durometer material and one or more inner layer(s) of successively lower durometer materials which has a compliance not substantially greater than (e.g., not more than about 10% to about 20% greater than), and preferably about equal to a balloon which is formed of 100% of the highest durometer material but which has a larger wall thickness than the multilayered balloon of the invention. This balloon has a very thin total wall thickness provides an improved low profile and flexibility due to the thinner walls of the balloon, but, in accordance with the invention, nonetheless continues to provide a low compliance despite the thin wall.

The rupture pressure and compliance of a balloon are affected by the strength (e.g., hoop strength) of a balloon. Because a softer material generally has a relatively lower hoop strength, the presence of the lower durometer material forming the inner layer(s) of the balloon is not generally expected to provide a relatively higher modulus balloon. However, a multilayered balloon preferably has a higher modulus than, and a rupture pressure which is not substantially less than, a balloon formed of 100% of the highest durometer material.

The presence of the lower durometer material inner layer(s) does provide layers of increased softness, and therefore preferably provides a balloon that is softer and more flexible than a balloon formed of 100% of the highest durometer material. The multilayered balloon can be made from elastomers, which typically have a lower flexural modulus than nonelastomers. Elastomeric polymers suitable for forming the first and/or second layer of the multilayered balloon typically have a flexural modulus of about 40 kpsi to about 110 kpsi. Thus, unlike nonelastomeric materials such as PET which have been used in the past to provide relatively low compliance catheter balloons, the multilayered noncompliant balloon is preferably formed of one or more elastomers which provide for improved balloon flexibility.

The balloon catheter also can be at least partially loaded with therapeutic agent which is allowed to treat the walls of the body vessel. “Therapeutic agent” as used herein, refers to any compound, mixture of compounds, or composition of matter consisting of a compound, which produces a therapeutic or useful result. The therapeutic agent can be a polymer, a marker, such as a radiopaque dye or particles, or can be a drug, including pharmaceutical and therapeutic agents, or an agent including inorganic or organic drugs without limitation. The agent or drug can be in various forms such as uncharged molecules, components of molecular complexes, pharmacologically acceptable salts such as hydrochloride, hydrobromide, sulfate, laurate, palmitate, phosphate, nitrate, borate, acetate, maleate, tartrate, oleate, and salicylate.

An agent or drug that is water insoluble can be used in a form that is a water-soluble derivative thereof to effectively serve as a solute, and on its release from the device, is converted by enzymes, hydrolyzed by body pH or metabolic processes to a biologically active form. Additionally, the agents or drug formulations can have various known forms such as solutions, dispersions, pastes, particles, granules, emulsions, suspensions and powders. The drug or agent may or may not be mixed with polymer or a liquid as desired.

In an embodiment of the invention, at least one therapeutic agent can be selected from but not limited to anti-proliferative, anti-inflammmatory, antineoplastic, antiplatelet, anti-coagulant, anti-fibrin, antithrombotic, antimitotic, antibiotic, antiallergic and antioxidant compounds. Thus, the therapeutic agent can be, again without limitation, a synthetic inorganic or organic compound, a protein, a peptide, a polysaccharides and other sugars, a lipid, DNA and RNA nucleic acid sequences, an antisense oligonucleotide, an antibodies, a receptor ligands, an enzyme, an adhesion peptide, a blood clot agent including streptokinase and tissue plasminogen activator, an antigen, a hormone, a growth factor, a ribozyme, a retroviral vector, an anti-proliferative agent including rapamycin (sirolimus), 40-O-(2-hydroxyethyl)rapamycin (everolimus), 40-O-(3-hydroxypropyl)rapamycin, 40-O-(2-hydroxyethyoxy)ethylrapamycin, 40-O-tetrazolylrapamycin (zotarolimus, ABT-578), paclitaxel, docetaxel, methotrexate, azathioprine, vincristine, vinblastine, fluorouracil, doxorubicin hydrochloride, and mitomycin, an antiplatelet compound, an anticoagulant, an antifibrin, an antithrombins including sodium heparin, a low molecular weight heparin, a heparinoid, hirudin, argatroban, forskolin, vapiprost, prostacyclin, a prostacyclin analogue, dextran, D-phe-pro-arg-chloromethylketone (synthetic antithrombin), dipyridamole, glycoprotein IIb/IIIa platelet membrane receptor antagonist antibody, recombinant hirudin, a thrombin inhibitor including Angiomax ä, a calcium channel blocker including nifedipine, colchicine, a fibroblast growth factor (FGF) antagonist, fish oil (omega 3-fatty acid), a histamine antagonist, lovastatin, a monoclonal antibodie, nitroprusside, a phosphodiesterase inhibitor, a prostaglandin inhibitor, suramin, a serotonin blocker, a steroid, a thioprotease inhibitor, triazolopyrimidine, a nitric oxide or nitric oxide donor, a super oxide dismutase, a super oxide dismutase mimetic, estradiol, an anticancer agent, a dietary supplement including vitamins, an anti-inflammatory agent including aspirin, tacrolimus, dexamethasone and clobetasol, a cytostatic substance including angiopeptin, an angiotensin converting enzyme inhibitor including captopril, cilazapril or lisinopril, an antiallergic agent including permirolast potassium, alpha-interferon, bioactive RGD, and genetically engineered epithelial cells. Other therapeutic agents which are currently available or that can be developed in the future for use with implantable medical devices can likewise be used and all are within the scope of this invention.

Examples of such antithrombotics, anticoagulants, antiplatelet agents, and thrombolytics include sodium heparin, low molecular weight heparins, heparinoids, hirudin, argatroban, forskolin, vapriprost, prostacyclin and prostacylin analogues, dextran, D-phe-pro-arg-chlorometh-ylketone (synthetic antithrombin), dipyridamole, glycoprotein IIb/IIIa (platelet membrane receptor antagonist antibody), recombinant hirudin, and thrombin inhibitors such as Angiomax™, from Biogen, Inc., Cambridge, Mass.; and thrombolytic agents, such as urokinase, e.g., Abbokinase™ from Abbott Laboratories Inc., North Chicago, Ill., recombinant urokinase and pro-urokinase from Abbott Laboratories Inc., tissue plasminogen activator (Alteplase™ from Genentech, South San Francisco, Calif. and tenecteplase (TNK-tPA).

Examples of such cytostatic or antiproliferative agents include rapamycin and its analogs such as everolimus, ABT-578, i.e., 3S,6R,7E,9R,10R,12R,14S,15E,17E,19E,21S,23S,26R,27R,34aS)-9,10,12,13,14,2-1,22,23,24,25,26,27,32,33,34,34a-Hexadecahydro-9,27-dihydroxy-3-[(1R)-2-[(-1S,3R,4R)-3-methoxy-4-tetrazol-1-yl)cyclohexyl]-1-methylethyl]-10,21-dimet-hoxy-6,8,12,14,20,26-hexamethyl-23,27-epoxy-3H-pyrido[2,1-c][1,4]oxaazacyclohentriacontine-1,5,11,28,29(4H,6H,31H)-pentone; 23,27-Epoxy-3H pyrido[2,1-c][1,4]oxaazacyclohentria-contine-1,5,11,28,29(4H,6H,31H)-pento-ne, which is disclosed in U.S. Pat. No. 6,015,815, U.S. Pat. No. 6,329,386, U.S. Publication 2003/129215, filed on Sep. 6, 2002, and U.S. Publication 2002/123505, filed Sep. 10, 2001, the disclosures of which are each incorporated herein by reference thereto, tacrolimus and pimecrolimus, angiopeptin, angiotensin converting enzyme inhibitors such as captopril, e.g, Capoten® and Capozide® from Bristol-Myers Squibb Co., Stamford, Conn., cilazapril or lisinopril, e.g., Prinivil™ and Prinzide™ from Merck & Co., Inc., Whitehouse Station, N.J.; calcium channel blockers such as nifedipine, amlodipine, cilnidipine, lercanidipine, benidipine, trifluperazine, diltiazem and verapamil, fibroblast growth factor antagonists, fish oil (omega 3-fatty acid), histamine antagonists, lovastatin, e.g. Mevacor™ from Merck & Co., Inc., Whitehouse Station, N.J. In addition, topoisomerase inhibitors such as etoposide and topotecan, as well as antiestrogens such as tamoxifen can be used.

Examples of such anti-inflammatories include colchicine and glucocorticoids such as betamethasone, cortisone, dexamethasone, budesonide, prednisolone, methylprednisolone and hydrocortisone. Non-steroidal anti-inflammatory agents include flurbiprofen, ibuprofen, ketoprofen, fenoprofen, naproxen, diclofenac, diflunisal, acetominophen, indomethacin, sulindac, etodolac, diclofenac, ketorolac, meclofenamic acid, piroxicam and phenylbutazone.

Examples of such antineoplastics include alkylating agents such as altretamine, bendamucine, carboplatin, carmustine, cisplatin, cyclophosphamide, fotemustine, ifosfamide, lomustine, nimustine, prednimustine, and treosulfin, antimitotics such as vincristine, vinblastine, paclitaxel, e.g., TAXOL® by Bristol-Myers Squibb Co., Stamford, Conn., docetaxel, e.g., Taxotere™ from Aventis S. A., Frankfurt, Germany, antimetabolites such as methotrexate, mercaptopurine, pentostatin, trimetrexate, gemcitabine, azathioprine, and fluorouracil, and antibiotics such as doxorubicin hydrochloride, e.g., Adriamycin™ from Pharmacia & Upjohn, Peapack, N.J., and mitomycin, e.g., Mutamycin™ from Bristol-Myers Squibb Co., Stamford, Conn., agents that promote endothelial cell recovery such as estradiol.

Other agents and materials could conceivably be delivered into a patient anatomy. For example, angiogenetic factors could be delivered. This includes growth factors such as isoforms of vasoendothelial growth factor (VEGF), fibroblast growth factor (FGF, e.g. beta-FGF), Del 1, hypoxia inducing factor (HIF 1-alpha), monocyte chemoattractant protein (MCP-1), nicotine, platelet derived growth factor (PDGF), insulin-like growth factor (HGF), estrogens, follistatin, proliferin, prostaglandin E1 and E2, tumor necrosis factor (TNF-alpha), interleukin 8 (Il-8), hematopoietic growth factors, erythropoietin, granulocyte-colony stimulating factors (G-CSF) and platelet-derived endothelial growth factor (PD-ECGF). In some embodiments, angiogenesis promoting factors include, but are not intended to be limited to, peptides, such as PR39, PR11 and angiogenin, small molecules, such as PHD inhibitors, or other agents, such as eNOS enhancers.

While the foregoing therapeutic agents are known for their preventive and treatment properties, the substances or agents are provided by way of example and are not meant to be limiting. Further, other therapeutic agents that are currently available or may be developed are equally applicable for use with the present invention.

If desired or necessary, the therapeutic agent can include a binder to carry, load, or allow sustained release of an agent, such as but not limited to a suitable polymer or similar carrier. The term “polymer” is intended to include a product of a polymerization reaction inclusive of homopolymers, copolymers, terpolymers, etc., whether natural or synthetic, including random, alternating, block, graft, branched, cross-linked, blends, compositions of blends and variations thereof. The polymer can be in true solution, saturated, or suspended as particles or supersaturated in the therapeutic agent. The polymer can be biocompatible, biosolvable, biostable, or biodegradable.

A variety of suitable self expanding stent designs can be used in a stent delivery system of the invention. Details regarding stent structure can be found in U.S. Pat. No. 6,709,454 (Cox et al.), U.S. Pat. No. 6,663,664 (Pacetti), U.S. Pat. No. 6,375,676 (Cox), U.S. Pat. No. 4,830,003 (Wolff et al.), and U.S. Pat. No. 4,580,568 (Gianturco), incorporated by reference herein in their entireties.

While described herein in terms of certain preferred embodiments, various modifications and improvements can be made to the invention without departing from the scope thereof. Additionally, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

We claim:

1. A stent delivery system, comprising:
a) a delivery catheter having an inner tubular member and an outer tubular member adapted for axial movement with respect to each other, such that the outer tubular member has an advanced configuration surrounding a distal section of the inner tubular member, and a proximally retracted configuration, and the inner tubular member is configured for proximally withdrawing the distal section of the inner tubular member into the outer tubular member in the retracted configuration and thereby transitioning the outer tubular member from the retracted to the advanced configuration;
b) a first stent and at least one second proximal stent, in a collapsed configuration in a space between the inner tubular member and outer tubular member with the outer tubular member in the advanced configuration, and configured to radially self expand from the collapsed configuration to an expanded configuration upon removal of a radially restraining force of the catheter outer tubular member, and the second collapsed stent is longitudinally spaced apart proximally from the first stent; and
c) a reversibly collapsible stent stop secured to the inner tubular member and slidably positionable to different locations relative to the collapsed stents in the delivery catheter, configured to radially collapse as the inner tubular member is proximally withdrawn into the outer tubular member and through the second collapsed stent, and to radially self expand along at least a section thereof at a location proximally adjacent to a proximal end of the second collapsed stent to an expanded configuration, such that the stent stop initially abuts a proximal end of the first collapsed stent to thereby inhibit proximal movement of the first stent in a first locational configuration, and is configured for being slidably positioned to abut the proximal end of the second collapsed stent to thereby inhibit proximal movement of the second stent in a second locational configuration.

2. The stent delivery system of claim 1 wherein the stent stop has at least a section with a conical shape with an outer surface tapering proximally from a larger outer diameter portion which abuts the proximal end of the stents to a smaller outer diameter proximal end section in the expanded configuration.

3. The stent delivery system of claim 2 wherein the stent stop has one or more slots in a distal end section and/or proximal section of a wall of the stent stop increasing the ability of the wall to reversibly radially expand to the same expanded diameter.

4. The stent delivery system of claim 2 wherein the conical section of the stent stop abutting the proximal end of the stents is a proximal section, and the stent stop includes a distal section having a smaller outer diameter than the larger outer diameter portion.

5. The stent delivery system of claim 4 wherein the distal section of the stent stop has a set outer diameter which does not radially expand or collapse on the inner tubular member.

6. The stent delivery system of claim 4 wherein the distal section of the stent stop has a distally tapering outer diameter.

7. The stent delivery system of claim 1 wherein the stent stop is formed of polymeric or metallic, or a combination of materials.

8. The stent delivery system of claim 1 wherein the stent stop is radiopaque.

9. The stent delivery system of claim 1 including a distal tip at a distal end of the inner tubular member, which is radiopaque and which has a radially enlarged section with an outer diameter substantially equal to an outer diameter of a distal end of the outer tubular member and an outer surface tapering distally from the radially enlarged section.

10. The stent delivery catheter of claim 1 wherein the stents have different lengths or maximum expanded diameters.

11. The stent delivery system of claim 1 including a third proximal stent in a space between the inner tubular member and outer tubular member proximal to the second proximal stent, configured to radially self expand from a collapsed configuration within the delivery catheter to an expanded configuration upon removal of a radially restraining force of the catheter outer tubular member.

12. The stent delivery catheter of claim 11 including second reversibly collapsible stent stop secured to the inner tubular member at a location proximal to said stent stop, radially self expanded along at least a section thereof proximally adjacent to the proximal end of the second collapsed stent with said stent stop in the first configuration, and configured to radially collapse as the inner tubular member is proximally withdrawn into the outer tubular member through the third collapsed stent and radially self expand along at least a section thereof at a location proximally adjacent to a proximal end of the third collapsed stent.

13. The stent delivery system of claim 1, further including a distal tip at the distal end of the inner tubular member which is adapted to retain a distal end of the stents.

14. A method of delivering and deploying self expanding stents in a patient's anatomy, comprising:
a) introducing within a body lumen of the patient a stent delivery catheter comprising
i) a delivery catheter having an inner tubular member and an outer tubular member adapted for axial movement with respect to each other, such that the outer tubular member has an advanced configuration surrounding a distal section of the inner tubular member, and a proximally retracted configuration, and the inner tubular member is configured for proximally withdrawing the distal section of the inner tubular member into the outer tubular member in the retracted configuration and thereby transitioning the outer tubular member from the retracted to the advanced configuration;
ii) a first stent and at least one second proximal stent, in a collapsed configuration in a space between the inner tubular member and outer tubular member with the outer tubular member in the advanced configuration, and configured to radially self expand from the collapsed configuration to an expanded configuration upon removal of a radially restraining force of the catheter outer tubular member, and the second collapsed stent is longitudinally spaced apart proximally from the first stent; and
iii) a reversibly collapsible stent stop secured to the inner tubular member and slidably positionable to different locations relative to the collapsed stents in the delivery catheter, configured to radially collapse as the inner tubular member is proximally withdrawn into the outer tubular member and through the second collapsed stent, and to radially self expand along at least a section thereof at a location proximally adjacent to a proximal end of the second collapsed stent to an expanded configuration, such that the stent stop initially abuts a proximal end of the first collapsed stent to thereby inhibit proximal movement of the first stent in a first locational configuration, and is configured for being slidably positioned to abut the proximal end of the second collapsed stent to thereby inhibit proximal movement of the second stent in a second locational configuration;
b) advancing the stent delivery system to position the first collapsed stent at a desired treatment site in the body lumen with the outer tubular member in the advanced configuration, and deploying the first stent by proximally retracting the outer member relative to the inner tubular member and first stent with the stent stop in the first locational configuration, such that the stent stop inhibits proximal movement of the first stent, so that the first stent radially self expands to a deployed configuration in the body lumen;
c) proximally withdrawing the distal section of the inner tubular member into the outer tubular member in the retracted configuration and thereby transitioning the outer tubular member from the retracted to the advanced configuration, and positioning the stent stop in the second locational configuration by collapsing the stent stop as the stent stop is proximally retracted through the collapsed second stent and radially self expanding the stent stop along at least a section thereof at a location proximally adjacent to the proximal end of the second collapsed stent.

15. The method of claim 14 including deploying the second stent at a desired treatment site in the patient's anatomy, either with or without repositioning the stent delivery system in the patient after deployment of the first stent, by proximally retracting the outer member relative to the inner tubular member and second stent, with the stent stop in the second locational configuration, such that the stent stop inhibits proximal movement of the second stent.

16. A stent delivery system, comprising:
a) a delivery catheter having an inner tubular member and an outer tubular member adapted for axial movement with respect to each other, such that the outer tubular member has an advanced configuration surrounding a distal section of the inner tubular member, and a proximally retracted configuration, and the inner tubular member is configured for proximally withdrawing the distal section of the inner tubular member into the outer tubular member in the retracted configuration and thereby transitioning the outer tubular member from the retracted to the advanced configuration;
b) a first stent and at least one second proximal stent, in a collapsed configuration in a space between the inner tubular member and outer tubular member with the outer tubular member in the advanced configuration, and configured to radially self expand from the collapsed configuration to an expanded configuration upon removal of a radially restraining force of the catheter outer tubular member, and the second collapsed stent is longitudinally spaced apart proximally from the first stent; and
c) a first stent stop in abutting relationship with the first stent and an additional stent stop associated with each additional stent, each additional stent stops being in an abutting relationship with the additional stents.

17. The stent delivery system of claim 16, wherein each stent stop is placed on the inner tubular member.

18. The stent delivery system of claim 16, wherein each stent stop is formed on an inner surface of the outer tubular member.

19. The stent delivery system of claim 16, wherein at least one stent stop is formed on an inner surface of the outer tubular member and at least one stent stop is placed on the inner tubular member.

20. A stent delivery catheter, comprising:
an elongate shaft member;
an expandable member associated with the elongate shaft member;
a stent in a collapsed configuration mounted to the expandable member; and
a pair of stent retainers, each having a stent retainer mounting portion secured to the elongate shaft member and an engaging portion slidingly disposed on a portion of the expandable member, such that each stent retainer initially engages an end of the collapsed stent to thereby inhibit movement of the stent from its mounted position.

21. The stent delivery catheter of claim 20, wherein the stent retainer engages the stent by abutting against the proximal end of the stent.

22. The stent delivery catheter of claim 20, wherein the stent retainer engages the stent by having a portion of the retainer placed over the proximal end of the stent.

23. The stent delivery catheter of claim 20, wherein the proximal portion of the stent retainer is frictionally secured to the elongate shaft member.

24. The stent delivery catheter of claim 23, wherein the stent retainer is made from a rigid or semi rigid material it moves longitudinally away from the expandable member as the expandable member overcomes the stent retainer retaining friction.

25. The stent delivery catheter of claim 20, wherein the stent retainer includes at least a pair of cylindrical rings attached together which are adapted to expand with the expandable member as it expands the mounted stent.

26. The stent delivery catheter of claim 25, wherein the rings of the stent retainer move with the expandable member as it partially expands to continue to engage the stent and inhibit proximal and distal movement of the stent from its mounted position.

27. The stent delivery catheter of claim 20, wherein the stent retainers are integrally formed on the expandable member.

28. The stent delivery catheter of claim 20, wherein the stent retainers are separate components which are attached to the expandable member.

29. A stent delivery catheter, comprising:
an elongate shaft member;
an expandable member associated with the elongate shaft member;
a plurality of struts forming a stent, the stent being mounted in a collapsed configuration on the expandable member; and
at least one stent retainer ring disposed on a portion of mounted stent such that the stent retainer ring inhibits proximal movement of the stent from its mounted position, the retainer ring having a width and a thickness, the ring having a plurality of tear sections which have a width which is less that the width of the remaining portion of the ring.

30. The stent delivery catheter of claim 29, wherein the retainer ring has a wall thickness and each of the tear sections have a wall thickness which is less that the wall thickness of the remaining portion of the retainer ring.

31. The stent delivery catheter of claim 29, wherein the tear sections of the retainer ring are adapted to be placed between struts of the stent as it is mounted to the expandable member.

32. The stent delivery catheter of claim 29, wherein the tear sections of the retainer ring are adapted to break when the expandable member is expanded.

33. The stent delivery catheter of claim 32, wherein at least a portion of the retainer ring is secured to the stent by a bioabsorbable material.
